(19) United States
(12) Patent Application Publication
(10) Pub. No.: US 2026/0027381 A1
(43) Pub. Date: Jan. 29, 2026

(54) DISPOSABLE LIGHT THERAPY HAND PROBES

(71) Applicant: GlobaLASEReach Inc., Manitowoc, WI (US)

(72) Inventors: Robert Nolan Hetz, Manitowoc, WI (US); Josh Zarecky, Fishers, IN (US); Joseph Pietrafitta, Manitowoc, WI (US); David Stoltzmann, Manitowoc, WI (US)

(21) Appl. No.: 19/280,360

(22) Filed: Jul. 25, 2025

Related U.S. Application Data
(60) Provisional application No. 63/675,684, filed on Jul. 25, 2024.

(51) Int. Cl. A61N 5/06; A61N 5/00

(52) U.S. Cl. CPC A61N 5/0603; A61N 2005/005; A61N 2005/0626; A61N 2005/063; A61N 2005/0632; A61N 2005/0644; A61N 2005/0666

(57) ABSTRACT

A handheld therapy device includes a handle having a fiber optic cable and a diffusing light assembly. The handheld therapy device further includes a beam transmission tube attached to the handle. The beam transmission tube is configured to receive diffused light from the diffusing light assembly and allow the diffused light to pass through the beam transmission tube in an axial direction. The handheld therapy device further includes a beam reflective end configured to receive the diffused light passed through the beam transmission tube and to reflect the diffused light in a non-axial direction. The handheld therapy device further includes a transparent tube removably attached to the handle and at least partially enveloping the beam transmission tube. The transparent tube is at least partially transparent to allow the diffused light reflected in the non-axial direction off of the beam reflective end to pass therethrough.

### CROSS-REFERENCE TO RELATED APPLICATION

This application claims the benefit of and priority to U.S. Patent Application No. 63/675,684, filed on Jul. 25, 2024, the entirety of which is incorporated by reference herein.

### BACKGROUND

The present disclosure relates to devices for delivering precision phototherapy, also known more specifically as photodynamic phototherapy or photobiomodulation therapy (“PBMT”). Light (photonic radiation) at certain wavelengths is more readily absorbed by molecules in certain tissues, identified as “chromophores,” which in turn can stimulate or retard certain metabolic processes. This can include stimulating, suppressing, or denaturing cellular tissues, interstitial tissues, and intracellular tissue components. The deliberate exposure of tissues to light for this purpose is known as “phototherapy,” “photobiomodulation therapy,” “low level light therapy,” “photodynamic therapy,” or “laser physiotherapy” in various applications.

### SUMMARY

One embodiment relates to a handheld probe device. The handheld therapy device includes a handle having a fiber optic cable and a diffusing light assembly. The fiber optic cable extends through the handle and is configured to transmit a beam of coherent light through the diffusing light assembly. The handheld therapy device further includes a beam transmission tube attached to the handle. The beam transmission tube is configured to receive diffused light from the diffusing light assembly and allow the diffused light to pass through the beam transmission tube in an axial direction. The handheld therapy device further includes a beam reflective end removably attached to a distal end of the beam transmission tube. The beam reflective end is configured to receive the diffused light passed through the beam transmission tube and to reflect the diffused light in a non-axial direction. The handheld therapy device further includes a transparent tube removably attached to the handle and at least partially enveloping the beam transmission tube. The transparent tube is at least partially transparent to allow the diffused light reflected in the non-axial direction off of the beam reflective end to pass therethrough.

Another embodiment relates to a handheld probe device. The handheld therapy device includes a handle having a fiber optic cable and a diffusing light assembly. The fiber optic cable extends through the handle and is configured to transmit a beam of coherent light through the diffusing light assembly. The handheld therapy device further includes a beam delivery cone removably attached to the handle. The beam delivery cone is configured to receive diffused light from the diffusing light assembly and allow the diffused light to pass axially through the beam delivery cone. The handheld therapy device further includes a transparent emission cap removably attached to a distal end of the beam delivery cone. The transparent emission cap is at least partially transparent to allow the diffused light passing axially through the beam delivery cone to pass therethrough.

Another embodiment relates to a handheld probe device. The handheld probe device includes a handle having a fiber optic cable and a diffusing light assembly. The fiber optic cable extends through the handle and is configured to transmit a beam of coherent light through the diffusing light assembly. The handheld therapy device further includes a beam passing component removably attached to the handle. The beam passing component is at least partially hollow and is configured to receive diffused light from the diffusing light assembly and allow the diffused light to pass through the beam passing component in an axial direction. The handheld therapy device further includes a transparent emission component removably attached to one of the handle or a distal end of the beam passing component. The transparent emission component is at least partially transparent to allow the diffused light passing axially through the beam passing component to pass therethrough.

### BRIEF DESCRIPTION OF THE DRAWINGS

Further features, characteristics, and advantages of the present disclosure will become apparent to a person of ordinary skill in the art from the following detailed description of embodiments of the present disclosure, made with reference to the drawings annexed, in which like reference characters refer to like elements.

FIG. 1 shows an encapsulating tube non-cooled device, according to an example embodiment.

FIG. 2 shows an exploded view of the encapsulating tube non-cooled device of FIG. 1, according to an example embodiment.

FIG. 3A shows a front view of the encapsulating tube non-cooled device of FIG. 1, according to an example embodiment.

FIG. 3B shows a cross-section of the encapsulating tube non-cooled device of FIG. 3A, taken along section line A-A, according to an example embodiment.

FIG. 4 shows a detail view of a diffusing lens of FIG. 3B, taken along detail line -B-, according to an example embodiment.

FIG. 5 shows a detail view of a beam reflective end of FIG. 3B, taken along detail line -C-, according to an example embodiment.

FIGS. 6A and 6B show a first beam reflective end design configuration, according to example embodiments.

FIGS. 6C and 6D show a second beam reflective end design configuration, according to example embodiments.

FIG. 7A shows a first beam profile of the first beam reflective end design configuration shown in FIGS. 6A and 6B, according to an example embodiment.

FIG. 7B shows a second beam profile of the second beam reflective end design configuration shown in FIGS. 6C and 6D, according to an example embodiment.

FIG. 8A shows a section view of an inline projection device with no reflector, according to an example embodiment.

FIG. 8B shows a beam profile of the inline projection device of FIG. 8A, according to an example embodiment.

FIG. 9A shows a section view of an inline projection device having a beam forming optics configuration including a double concave beam expansion, according to an example embodiment.

FIG. 9B shows a beam profile of the inline projection device of FIG. 9A, according to an example embodiment.

FIG. 10A shows a beam forming optics configuration including double convex lenses, according to an example embodiment.

FIG. 10B shows a beam profile of the beam forming optics configuration of FIG. 10A, according to an example embodiment.

FIG. 11A shows a beam forming optics configuration including a ball lens, according to an example embodiment.

FIG. 11B shows a beam profile of the beam forming optics configuration of FIG. 11A, according to an example embodiment.

FIG. 12A shows a first encapsulating tube cover, according to an example embodiment.

FIG. 12B shows a second encapsulating tube cover, according to an example embodiment.

FIG. 13 shows a non-cooled surface projection device, according to an example embodiment.

FIG. 14A shows a top view of the non-cooled surface projection device of FIG. 13, according to an example embodiment.

FIG. 14B shows a cross-section of the non-cooled surface projection device of FIG. 14A, taken along section line D-D, according to an example embodiment.

FIG. 15 shows a detail view of the non-cooled surface projection device of FIG. 14B, taken along detail line -E-, according to an example embodiment.

FIG. 16 shows a cooled encapsulating tube device, according to an example embodiment.

FIG. 17 shows an exploded view of the cooled encapsulating tube device of FIG. 16, according to an example embodiment.

FIG. 18A shows a top view of the cooled encapsulating tube device of FIG. 16, according to an example embodiment.

FIG. 18B shows a cross-section of the cooled encapsulating tube device of FIG. 18A, taken along section line F-F, according to an example embodiment.

FIG. 19A shows a rear view of a cooling collar and beam tube, according to an example embodiment.

FIG. 19B shows a cross-section of the cooling collar and beam tube of FIG. 19A, taken along section line G-G, according to an example embodiment.

FIG. 20 shows a detail view of the cooling collar and beam tube of FIG. 19B, taken along detail line -H-, according to an example embodiment.

FIG. 21A shows a rear view of the cooled beam tube of FIG. 19A, according to an example embodiment.

FIG. 21B shows a cross-section of the cooled beam tube of FIG. 21A, taken along section lines J-J, according to an example embodiment.

FIG. 21C shows a cross-section of the cooled beam tube of FIG. 21A, taken along section lines K-K, according to an example embodiment.

FIG. 21D shows a cross-section of the cooled beam tube of FIG. 21A, taken along section lines L-L, according to an example embodiment.

FIG. 22 shows a cooled surface projection device, according to an example embodiment.

FIG. 23A shows a top view of the cooled surface projection device of FIG. 22, according to an example embodiment.

FIG. 23B shows a cross-section of the cooled surface projection device of FIG. 23A, taken along section line M-M, according to an example embodiment.

FIG. 24 shows a detail view of the cooled surface projection device of FIG. 23B, taken along detail line -N-, according to an example embodiment.

FIG. 25A shows front view of the cooled surface projection device of FIG. 22, according to an example embodiment.

FIG. 25B shows a detail view of the cooled surface projection device of FIG. 25A, taken along detail line -P-, according to an example embodiment.

FIG. 26A shows a rear view of a cooled beam cone and collar, according to an example embodiment.

FIG. 26B shows a side view of the cooled beam cone and collar of FIG. 26A, according to an example embodiment.

FIG. 26C shows a front view of the cooled beam cone and collar of FIG. 26A, according to an example embodiment.

FIG. 26D shows a section view of the cooled beam cone and collar of FIG. 26B, taken along section line Q-Q, according to an example embodiment.

FIG. 27A shows a side view of the cooled beam cone of FIG. 26A, according to an example embodiment.

FIG. 27B shows a cross-section of the cooled beam cone of FIG. 27A, taken along section line R-R, according to an example embodiment.

FIG. 28A shows a surface projection device ergonomic variation, according to an example embodiment.

FIG. 28B shows another surface projection device ergonomic variation, according to an example embodiment.

FIG. 29 shows a cooled tube device including a temperature sensor, according to an example embodiment.

FIG. 30A shows a front view of the cooled tube device of FIG. 29, according to an example embodiment.

FIG. 30B shows a cross-section of the cooled tube device of FIG. 30A, taken along section view S-S, according to an example embodiment.

FIG. 31 shows a detail view of the cooled tube device of FIG. 30B, taken along detail line -T-, according to an example embodiment.

FIG. 32A shows a side view of the cooled tube device of FIG. 29, shown with no tube, according to an example embodiment.

FIG. 32B shows a cross-section view of the cooled tube device of FIG. 32A, taken along section line U-U, according to an example embodiment.

FIG. 33 shows a detail view of the cooled tube device of FIG. 32B, taken along detail line -V-, according to an example embodiment.

FIG. 34 shows a detail view of the cooled tube device of FIG. 32A, taken along detail line -W-, according to an example embodiment.

FIG. 35A shows a transparent tube with temperature sensor integration, according to an example embodiment.

FIG. 35B shows a cross-section view of the transparent tube with temperature sensor integration of FIG. 35A, taken along section line X-X, according to an example embodiment.

FIG. 35C shows a detail view of the transparent tube with temperature sensor integration of FIG. 35B, taken along detail line -Y-, according to an example embodiment.

### DETAILED DESCRIPTION

The devices and embodiments detailed herein are configured for the administration of photo biomodulation therapy. For the purposes of this disclosure, references to external sources of coherent light utilized for the delivery of therapeutic photon energy and/or cooling systems for cooling phototherapy devices refer to the same or similar sources and/or systems as those disclosed in U.S. Pat. No. 11,318,323, entitled “Device for Delivering Precision Phototherapy, filed Aug. 21, 2020, and P.C.T. Application Publication No. WO2023/076264, entitled “Systems and Methods for Cooling a Phototherapy Device, filed, Oct. 25, 2022, the entireties of which are incorporated herein by reference.

The consumable cover devices described herein provide a variety of benefits for treating pain management, with applications encompassing transvaginal, transcutaneous and transrectal modalities. These devices provide an effective approach to addressing neuropathic pain, characterized by, for example, abnormal nerve signaling, and other pain types, such as nociceptive and central sensitization pain. Traditional treatments often yield limited results for these conditions. By providing targeted relief with minimal discomfort, the devices disclosed herein may provide improved therapeutic results that may be widely applicable for a variety of pain management strategies across various applications. Additionally, the consumable device interfaces described herein provide enhanced accessibility and safety in treatment delivery. Through these advancements, the devices described herein allow for improved pain management and outcomes for patients.

The following disclosure provides a variety of handheld devices for the delivery of photo biomodulation therapy that utilize a consumable component for contact to the sample or patient tissue surface. It will be appreciated that the consumable components are generally disposable, cleanable, and/or sterilizable between uses.

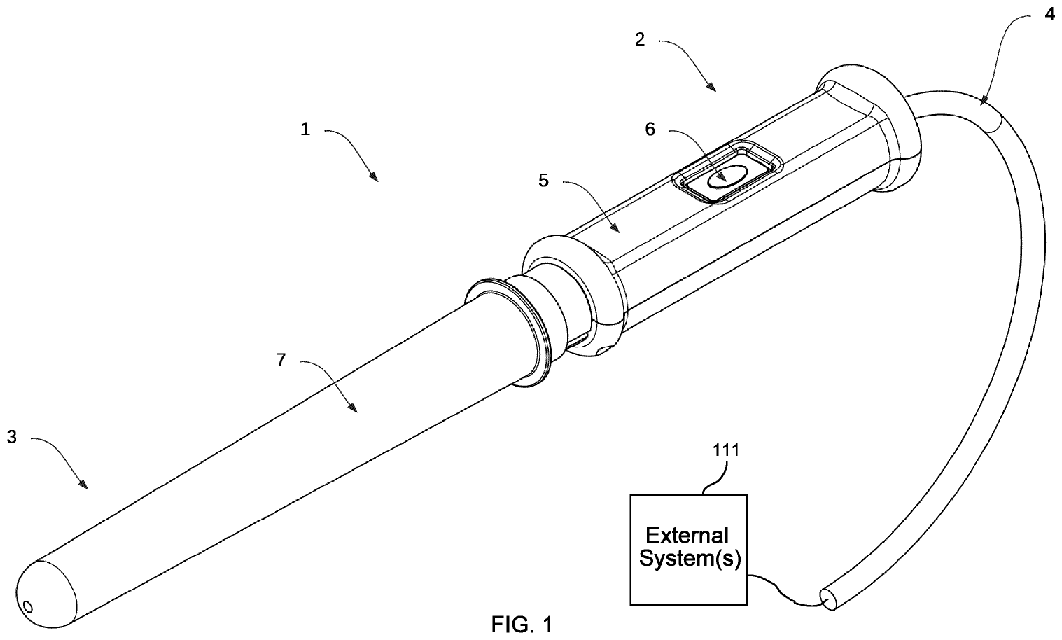

Referring to FIG. 1, a handheld consumable non-cooled therapy device, shown as handheld therapy device 1, is shown in a perspective view that depicts a complete assembly of the device with a consumable component, shown as a transparent tube 7, at the distal end 3 of the handheld therapy device 1. A proximal end 2 of the non-cooled therapy device 1 acts as a user holding and operation interface, referred to herein as a handle 5. The handle 5 has an integrated button 6 and a fiber optic cable (FOC) 4 extending from the proximal end 2 to one or more external systems 111. The one or more external systems 111 may comprise, for example, a therapeutic photon energy source, commonly a fiber coupled diode laser, another coherent light generator or light generation source, a cooling system, a measurement system (e.g., configured to measure and/or otherwise monitor sensor data), a computing control system, and/or any other system configured to interact with the handheld therapy device 1.

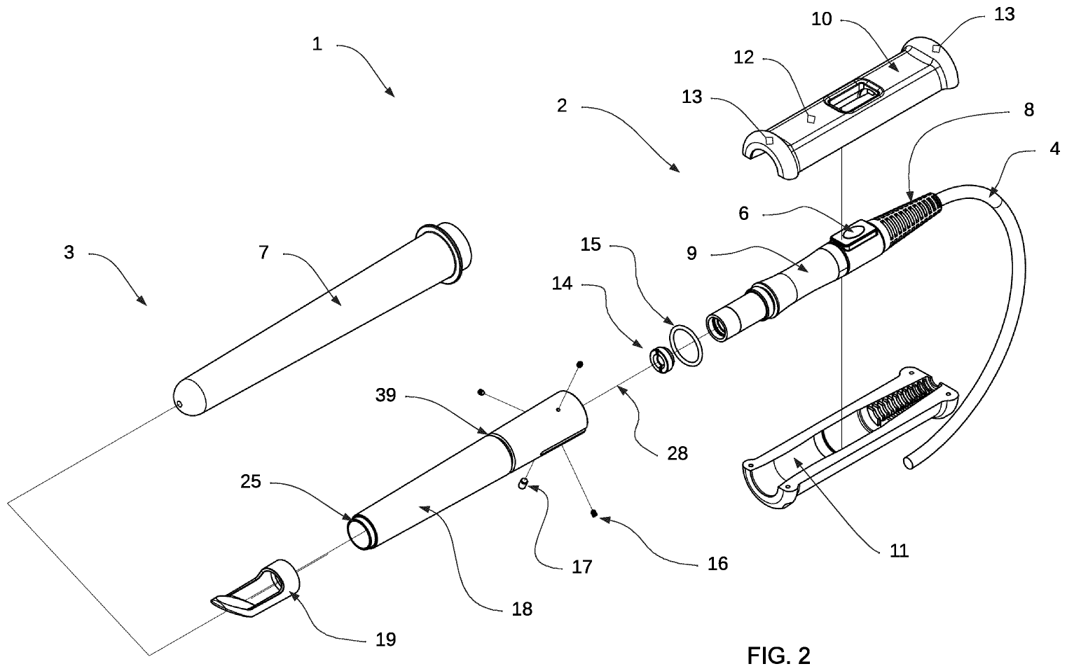

Referring to FIG. 2, an exploded view of the handheld therapy device 1 depicting the internal components is shown. The handle 5 (shown in FIG. 1) is located at the proximal end 2 of the handheld therapy device 1 and includes a functional core 9 which provides an interface between the FOC 4, the user button 6, a diffusing lens assembly 14, and the physical surfaces of the handle 5 that the user touches and holds, which are depicted as a clam shell assembly having a top component 10 and a bottom component 11 which surround the functional core 9 and form halves of a housing of the handle 5. Coupled to the distal end of the functional core 9 is the beam transmission tube 18 which serves as both a structure and a protected optical pathway for therapeutic photon energy emitted from the FOC 4 along a beam emission axis 28, through the beam diffusing lens assembly 14 to a beam reflective end 19, which reflects, directs, and shapes the photon light energy through the transparent tube 7 to a targeted treatment area. A strain relief 8 on the functional core 9 protects a connection between the FOC 4 and functional core 9 from wearing, fraying, etc.

The top component 10 has an orientation feature 12 depicted as a flat surface and end catch features 13 configured to improve grip and user handling. The top component 10 and the bottom component 11 have various structural features to accommodate encapsulating the functional core 9 including the button 6, the strain relief 8, and/or any other features and form needed to facilitate varying industrial designs for the device. The top component 10 and the bottom component 11 may be connected either removably or permanently using varying methods such as, for example, fasteners, adhesive, ultrasonic welding, interference fits between them, etc. In some embodiments, the handle 5 (shown FIG. 1) could be one integrated part or assembly where there is no separate core 9, top component 10, or bottom component 11.

When the handheld therapy device 1 is assembled, orientation features(s) 12 on the top component 10 are configured to be aligned with respect to the beam reflective end 19 to indicate to the operator the orientation of the distal end 3 when inserted for treatment in a cavity.

In some embodiments, the beam transmission tube 18 is removably attached to the functional core 9 and secured by one or more retention set screw(s) 16. Additionally, in some instances, a tube latch pin 17 located near a proximal end of the beam transmission tube 18 is configured to interface with a feature of the transparent tube 7 to retain and control orientation/rotation of the tube 7 with respect to the remainder of the handheld therapy device 1. Accordingly, in some instances, the beam transmission tube 18 and the transparent tube 7 are each individually removable from the handheld therapy device 1, such that either component may be selectively swapped out, as desired for a given application.

In some embodiments, the beam transmission tube 18 may include one or more o-ring(s) 15 and paired groove(s) 39 on the outside diameter of the beam tube 18 and configured to seal against the internal surface of the transparent tube 7.

In some embodiments, the beam reflective end 19 is coupled via a reflector interface 25 to the beam transmission tube 18. The reflector interface 25 may be permanent or separable. In some instances, the reflector interface 25 may be a strictly structural attachment. In some other instances, the reflector interface 25 may comprise one or more connections for electrical and/or pneumatic systems between the beam tube 18 and the beam reflective end 19. In some embodiments, the beam tube 18 and the reflective end 19 may be integrated as one piece.

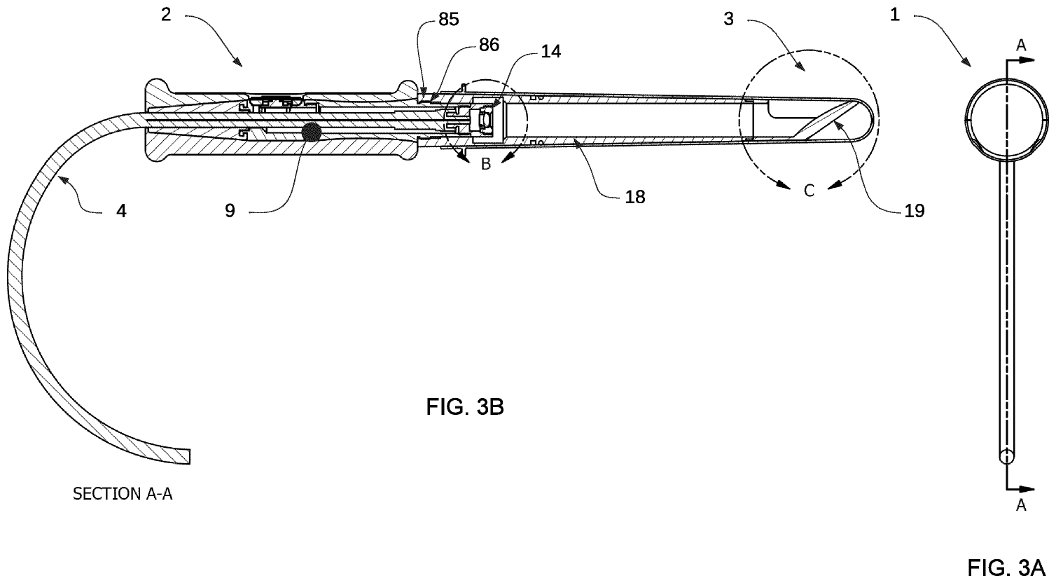

Referring to FIGS. 3A and 3B, a front view (FIG. 3A) and section view (FIG. 3B) of the handheld therapy device 1 are shown. At the intersection between the proximal end 2 (e.g., the handle 5) and the distal end 3 (e.g., the transparent tube 7, the beam transmission tube 18) are an attachment interface 85 and a mounting interface 86 of the handheld therapy device 1. The mounting interface 86 is a feature or set of mating features within the structure of the functional core 9 configured to which facilitate secure, removable connection to the attachment interface 85 of the beam transmission tube 18. As illustrated, in some embodiments, a junction between the attachment interface 85 and the mounting interface 86 of may be threaded. In other embodiments, various other coupling methods may be utilized including, but not limited to, snap features, detents, quarter turn or camming connections, press fits, etc. In some embodiments, the attachment interface 85 and the mounting interface 86 may be omitted in favor of an all-in-one device (e.g., a single, integrated piece).

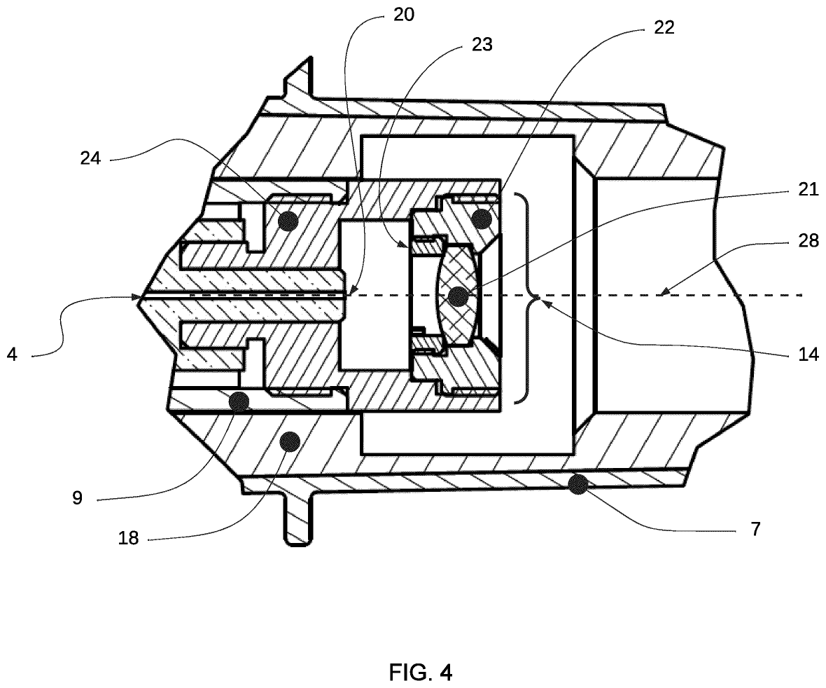

Referring to FIG. 4, a detail view of the handheld therapy device 1 is shown, illustrating various components and interfaces forming the diffusing lens assembly 14. The diffusing lens assembly 14 includes a beam diffusing lens 21, a lens retention nest 22, and a lens retainer 23. The beam diffusing lens 21 is located within a cavity of the lens retention nest 22 and is securely held in position by the lens retainer 23. In some embodiments, the lens retainer 23 may be threaded into the lens retention nest 22. However, in other embodiments, the lens retainer 23 may be coupled within the lens retention nest 22 using other methods, such as, for example, a compression fit, a snap-fit, adhesive, etc.

The diffusing lens assembly 14, which may be more generally referred to as beam forming optics as also referred to herein, is located on the beam emission axis 28 adjacent to an emission point 20 of the fiber optic cable 4 such that therapeutic photon energy (e.g., from a therapeutic light generation source of the external system 111) passes through the beam diffusing lens 21. It should be appreciated that the diffusing lens assembly 14 is just one example embodiment of beam forming optics. In various other embodiments, other beam forming optics described herein or other beam forming optics configurations generally may be utilized in place of the diffusing lens assembly 14.

The diffusing lens assembly 14 may be coupled to the functional core 9 via an optical positioning and alignment coupler referred to herein as a beam emission interface 24. The beam emission interface 24 is a physical junction between the functional core 9 and the diffusing lens assembly 14. In some embodiments, the beam emission interface 24 and lens retention nest 22 may be combined with the functional core 9 structure to form a single component.

The beam emission interface 24 is configured to control an axial alignment and offset distance between the emission point 20 (e.g., a distal end of the FOC 4) and the beam diffusing lens 21. The axial alignment and offset distance has a direct impact on the resulting beam profile and corresponding delivery of therapeutic photon energy.

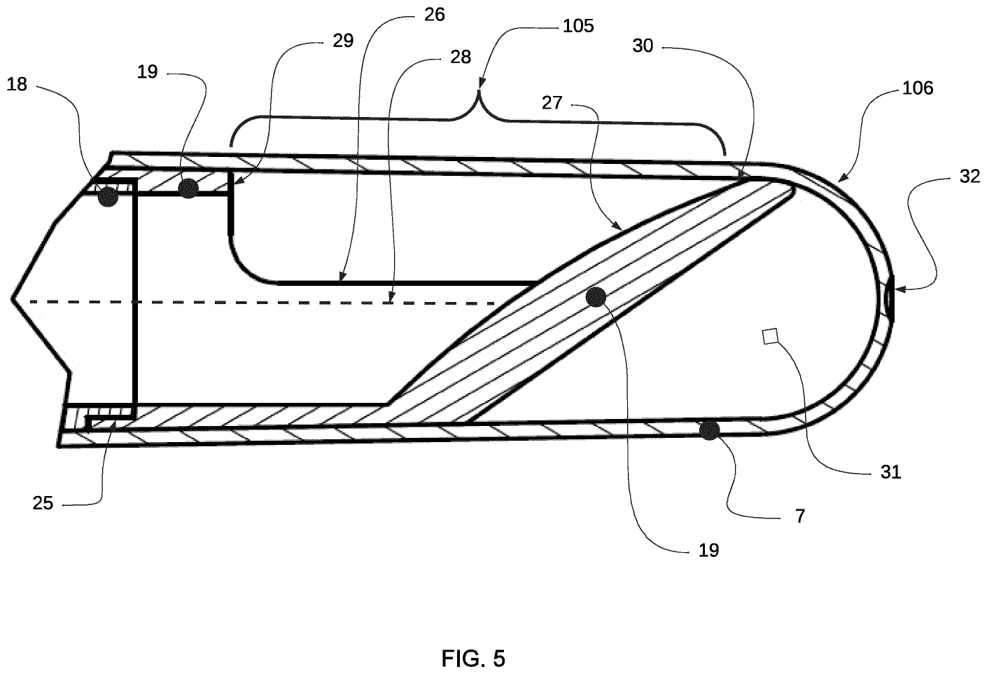

Referring to FIG. 5, a detail view illustrates various features and characteristics of the distal end 3 of the handheld therapy device 1. In some embodiments, the beam reflective end 19 is configured to reflect the therapeutic photon energy incident upon a convex reflector surface 27 orthogonal (or close to orthogonal) to the beam emission axis 28, such that the therapeutic photon energy is delivered to the patient tissue in contact with the exterior surface of the transparent tube 7. In some embodiments, the convex reflector surface 27 of the beam reflective end 19 is convex in both a first direction (e.g., distally with respect to emission axis 28) and a second direction perpendicular to the first direction (e.g., a directed extending perpendicular to the page with respect to FIG. 5). In some embodiments, a first radius of curvature of the convex reflector surface 27 of the beam reflective end 19 in the first direction is different than a second radius of curvature of the convex reflector surface 27 of the beam reflective end 19 in the second direction, as further discussed below.

A region of the transparent tube 7 through which the therapeutic photons are delivered to the patient is referred to herein as an optical window 105. The optical window 105 is a region or area of the transparent tube 7 within boundaries formed between an optical window proximal edge 29 and an optical window distal edge 30 along the beam emission axis 28 and between optical window wings 26. The optical window wings 26 radially limit the emission to only a portion of the circumference of the transparent tube 7. In the illustrated embodiment shown in FIG. 5, the optical window wings 26 are located at approximately 2 and 10 o'clock positions at the sides (e.g., where the optical window distal edge 30 would be the 12 o'clock position as depicted and where the axis of the referenced clock hand is concentric to the beam emission axis 28). This configuration produces an optical window 105 that is generally parallel to the beam emission axis 28 and open part way around the circumference of the transparent tube 7 between the proximal edge 29 and the distal edge 30. In some instances, the transparent tube 7 may only be partially transparent (e.g., the portion of the transparent tube 7 configured to be aligned with the optical window 105 to allow diffused light to pass therethrough), such that a remaining portion of the transparent tube 7 is opaque or translucent.

In some embodiments, the beam reflective end 19 is coupled directly to an end of the beam transmission tube 18 at a beam-tube-to-reflector interface 25. The profiles of the beam reflective end 19 and the beam transmission tube 18 are such that they fit snugly into a closed end 106 of the transparent tube 7. In the illustrated embodiment shown in FIG. 5, a body of the beam reflective end 19 is cut to remove material from the surface opposite the convex reflector 27 such that a void space 31 is created within the transparent tube 7 at the closed end 106.

In some embodiments, a sprue gate recess 32 may be included to facilitate manufacturing of the transparent tube 7 by injection molding from the tip of the transparent tube 7. The sprue gate recess 32 may serve to prevent plastic remnants from being a potential sharp point that could scratch, cut, or cause undue discomfort to a patient.

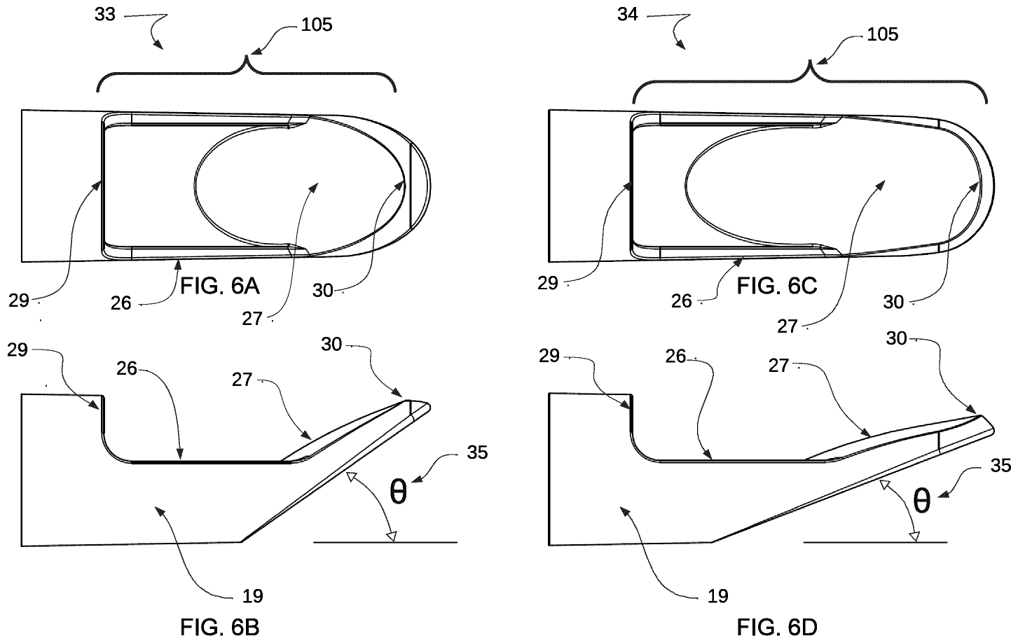

Referring to FIGS. 6A-6D, top and side views of two different potential beam reflective end examples depicting various potential characteristics of the beam reflective end 19 for different possible configurations and applications are shown. For example, FIGS. 6A and 6B show a perpendicular beam reflection end 33. Meanwhile, FIGS. 6C and 6D show a forward projected beam reflection end 34. It will be appreciated that the beam reflective end 19 can be configured as either the perpendicular beam reflection end 33 or as the forward projected beam reflection end 34. In some instances, the beam reflective end 19 can be configured with different characteristics, as desired for a given application.

The two configurations of the beam reflective end 19 shown in FIGS. 6A-6D represent different modes of therapeutic photon energy delivery configured to accommodate differing patient anatomy and targeted treatment locations. It should be appreciated that the optical window 105 through which the therapeutic photon energy is delivered may be varied or altered to accommodate these different configurations of the beam reflective end 19 by, for example, changing the locations of the proximal edge 29 and the distal edge 30 and/or adjusting the optical window wings 26 to increase or decrease the proportion of the reflective end 19 open to the optical window 105. The beam reflective end 19 may similarly be adjusted by modifying the profile curvature of the convex reflector surface 27 and/or varying an angle 35 of the convex reflective surface 27 of the beam reflective end 19 as desired for a given application.

In some embodiments, the convex reflective surface 27 may be a toroidal mirror whose profile is defined by two radii of curvature making up a section of a torus. In these embodiments, the resulting convex surface 27 may serve to selectively shape a spot profile by distributing the incident diffuse beam on the beam emission axis 28 in different proportions along the longitudinal and transverse axes of the convex reflective surface 27 by varying the two radii of curvature defining the topography of the convex reflective surface 27. In some embodiments, the radius of curvature along the longitudinal axis is different than the radius of curvature along the transverse axis.

The reflectance, transmittance, and emissivity of the convex reflective surface 27 affect and are configured to maximize treatment efficiency. In some embodiments, the reflective surface 27 is coated with gold and/or one or more protective layers to improve performance and/or durability.

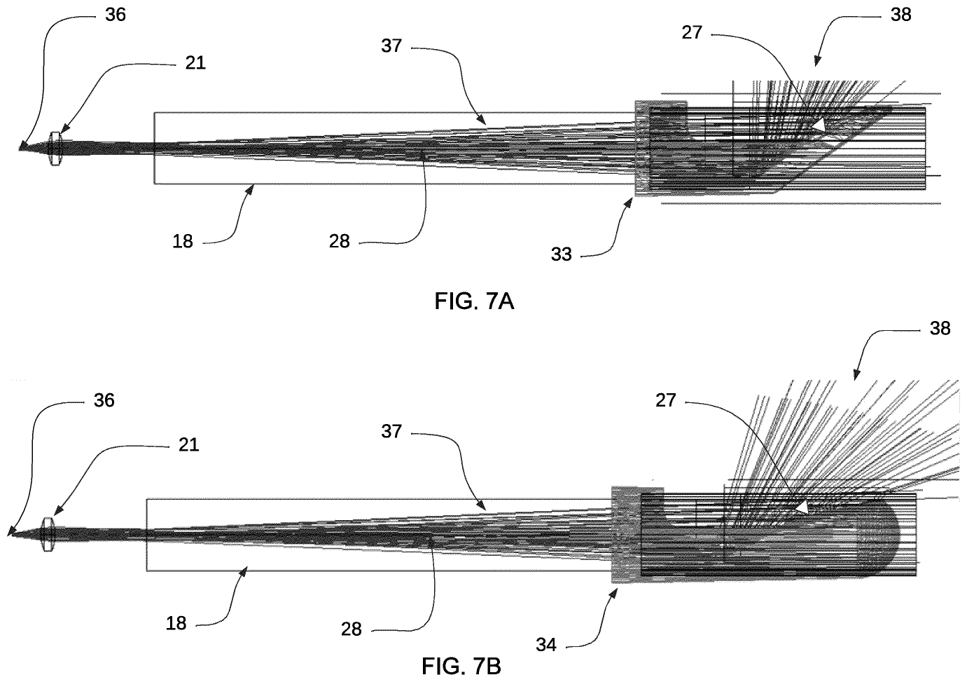

Referring to FIGS. 7A and 7B, optical ray trace illustrations depicting the difference between the perpendicular beam reflective end 33 and forward projected beam reflective end 34 are shown. In both cases the therapeutic photon energy emitted from the fiber optic cable 4 is depicted as the fiber emission beam profile 36 which is a slowly diverging Gaussian beam profile emitted on the beam emission axis 28. The fiber emission beam profile 36 passes through the beam diffusing lens 21, which smooths the Gaussian beam profile and produces a diverging beam with a uniform beam intensity across the profile referred to as a diffusing beam profile 37. The diffusing beam profile 37 projects down the length of the beam transmission tube 18 and onto the convex reflector surface 27 of the perpendicular beam reflection end 33 or the forward projected beam reflection end 34. The reflected beam profiles 38 illustrate the resulting difference between the perpendicular beam reflective end 33 and forward projected beam reflective end 34.

As shown in FIG. 7A, the reflected beam profile 38 produced by the perpendicular beam reflective end 33 is a slightly diverging profile of rays emitted approximately perpendicular to the beam emission axis 28. For example, as illustrated, at least some of the rays are emitted at approximately a 90 degree angle (e.g., between 95 degrees and 85 degrees) with respect to the beam emission axis 28. In some embodiments, this reflected beam profile 38 may be ideal, optimized, or otherwise suitable for treatment of the lower pelvic floor muscles. It will be appreciated that this reflected beam profile 38 may similarly be suitable for a variety of other treatment applications.

As shown in FIG. 7B, the reflected beam profile 38 produced by the forward projected beam reflective end 34 is generally a more dramatically diverging profile of rays emitted with forward distal projection at an angle of less than 90 degrees with respect to the beam emission axis 28 (e.g., the rays generally reflect at less than a 90 degree angle from their original trajectory down the beam transmission tube 18). In some embodiments, this reflected beam profile may be ideal, optimized, or otherwise suitable for treatment of the bladder. It will be appreciated that this reflected beam profile 38 may similarly be suitable for a variety of other treatment applications.

Regardless of whether the beam reflective end 19 is configured as the perpendicular beam reflective end 33 or forward projected beam reflective end 34, the convex reflector surface 27 is configured to reflect the diffusing beam profile 37, which may have a generally Gaussian distribution of energy, and produce the reflected beam profile 38 having a generally uniform distribution of energy.

As will be appreciated, the present disclosure provides a plurality of potential handheld therapy device embodiments. For simplicity, similar elements or components of each handheld therapy device (e.g., the handheld therapy device 1 discussed above, as well as handheld therapy device 201, 301, 401, 501, 701, 901 discussed below) will be labeled using similar reference numbers (e.g., beam diffusing lens 21, beam diffusing lens 221, beam diffusing lens 421). It should be appreciated that, apart from where the descriptions of certain elements or components of the various handheld therapy devices differ from one another (e.g., the descriptions contradict one another), similar elements having similar reference numbers may function similarly to each other, such that the description of an element or component of one handheld therapy device may be similarly applicable to a similar element or component of any other handheld therapy device described herein.

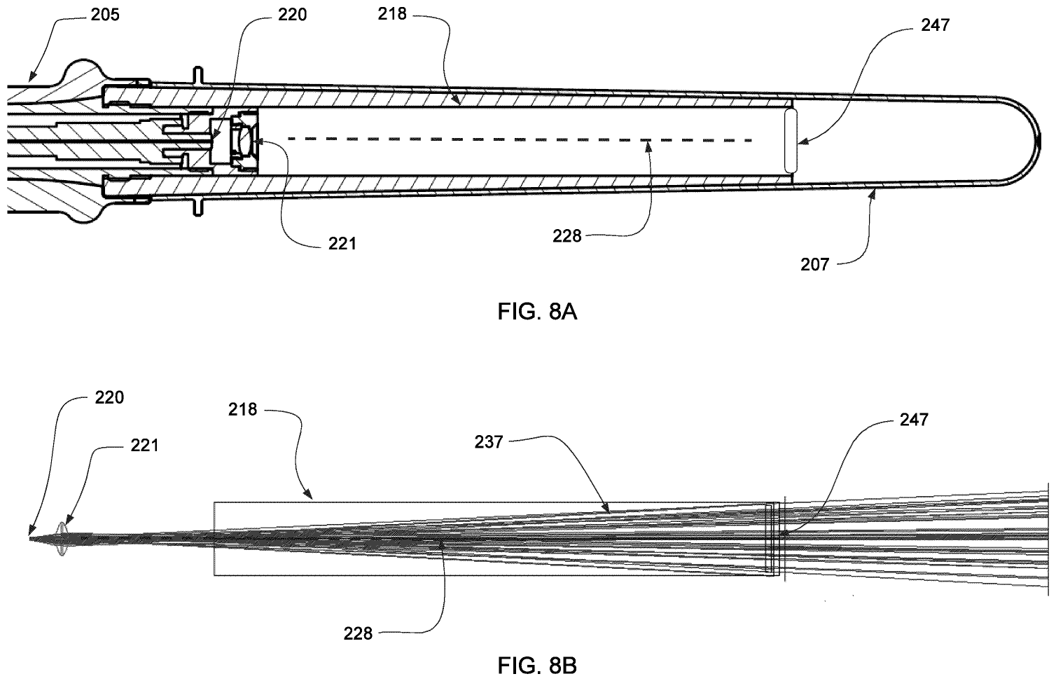

Referring to FIGS. 8A and 8B, another embodiment of a non-cooled encapsulating tube device, shown as handheld therapy device 201, is depicted as an inline projection device without a reflector. In this embodiment (i.e., the handheld therapy device 201), the coherent light emitted from an emission point 220 is diffused by a beam diffusing lens 221 and a resulting diffusing beam profile 237 continues along a beam emission axis 228 out of an end of a transparent tube 207.

In some instances, as shown in FIG. 8B, the beam transmission tube 218 of the handheld therapy device 201 may include a protective window 247 at the distal end of the beam transmission tube 218 within the transparent tube 207. The diffusing beam profile 237 produced by beam diffusing lens 221 would, if equipped, pass through the protective window 247 and be emitted through the distal end of the transparent tube 207. In some embodiments, the protective window 247 is made of a material that results in no (or negligible) optical power change to the diffusing beam profile 237.

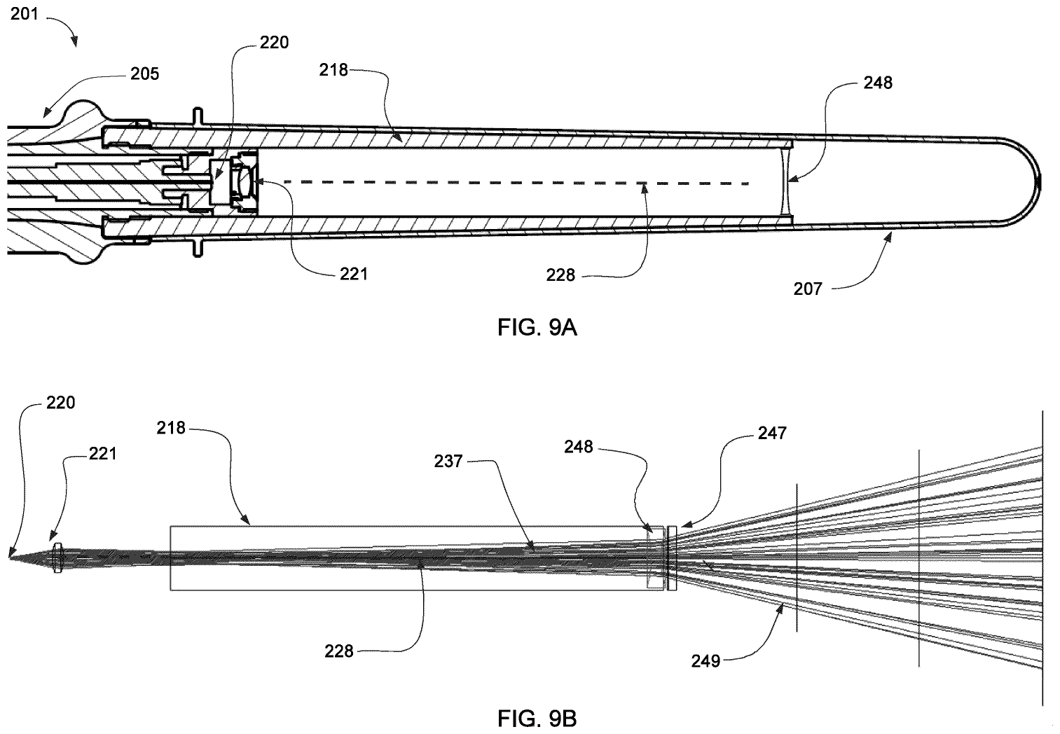

Referring to FIGS. 9A and 9B, the handheld therapy device 201 is depicted with an additional beam forming optical element, shown as a double concave lens 248, at the distal end of the beam transmission tube 218. As best shown in FIG. 9B, the light emitted at the emission point 220 is diffused through the beam diffusing lens 221, and then the diffusing beam profile 237 travels down the beam transmission tube 218, along the beam emission axis 228, where it passes through the double concave lens 248 and, in some embodiments, the protective window 247. The double concave lens 248 is configured to increase a rate of beam divergence producing an expanded beam profile 249 (shown in FIG. 9B). This arrangement of beam forming optics enables treatment of larger surfaces as a function of the size of the projected beam at a given distance.

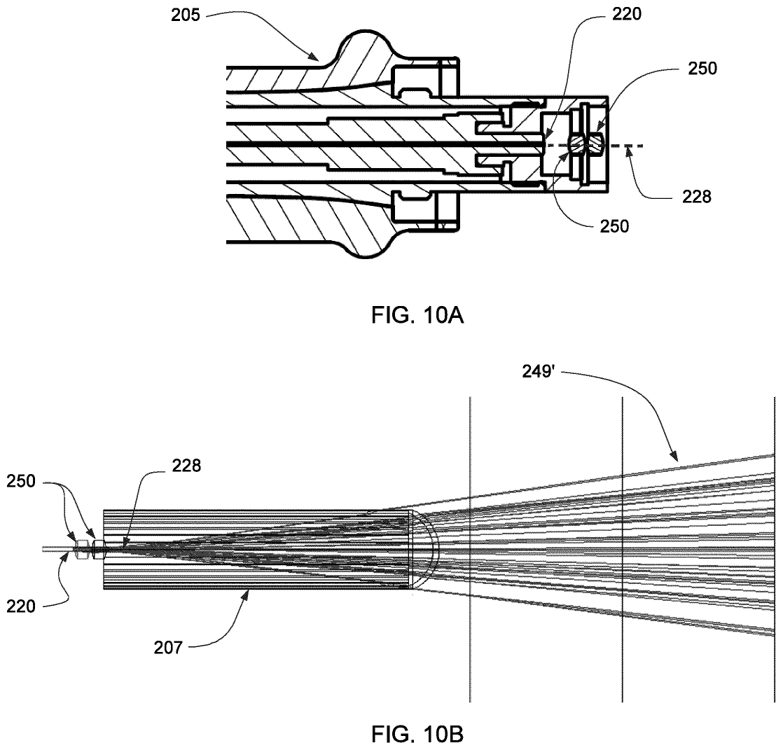

Referring to FIGS. 10A and 10B, another embodiment of beam forming optics is shown having a pair of double convex lenses 250 adjacent to the emission point 220. The pair of double convex lenses 250 is configured to produce a diverging diffused expanded beam profile 249′. In some embodiments, the diverging diffused expanded beam profile 249′ resulting from the beam forming characteristics of the double convex lenses 250 has a greater rate of divergence than the diffusing beam profile 237 of the beam diffusing lens 221 depicted in FIGS. 4 and 7A-9B. In some embodiments, this arrangement of beam forming optics enables treatment of a medium size area with a balance of complexity and efficiency.

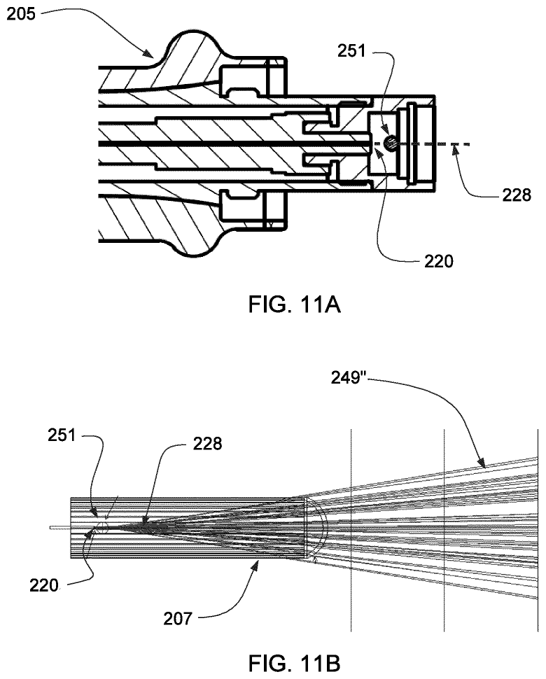

Referring to FIGS. 11A and 11B, another embodiment of beam forming optics is shown having a ball lens 251 adjacent to the emission point 220. The ball lens 251 produces a rapidly diverging diffused expanded beam profile 249”. In some embodiments, this arrangement of beam forming optics enables treatment of a maximum (e.g., a largest possible) area in the shortest possible projection distance with the least possible complexity (e.g., fewer components and dimensional requirements compared to other beam forming optics arrangements).

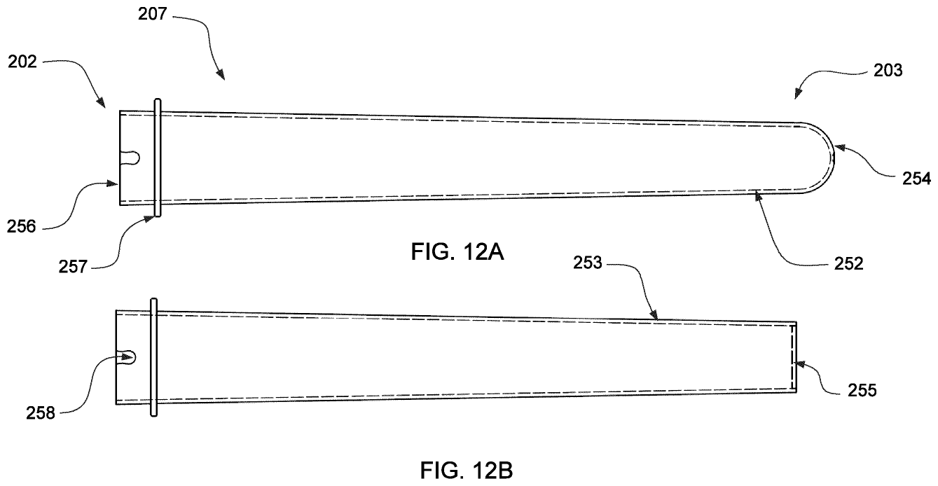

Referring to FIGS. 12A and 12B, additional embodiments of the transparent tube 207 are shown. Referring to both illustrated embodiments, the proximal end 202 of the transparent tube 207 has an open end 256 and includes a rib 257 and latch feature 258. In some embodiments, the transparent tube 207 serves as a bio barrier between internal components of the handheld therapy device 201 and patient tissue or biological fluids during treatment. In some embodiments, the transparent tube 207 is made of an optically clear material (e.g., a clear plastic, glass).

In some embodiments, the rib 257 serves as a limit of travel along the exterior wall 253, which may serve one or more functions including, for example, keeping a protective sleeve for the handle in place, serving as a depth reference for treatment applications where the encapsulating tube is inserted into a cavity, and/or as a physical depth limit.

In some embodiments, the interior wall 252 extends uniformly throughout the transparent tube 207 with respect to the exterior wall 253 from the open end 256 to the distal end 203. As illustrated in FIGS. 12A and 12B, the distal end 203 may take on various shapes and sizes to suit the needs of a particular application. The illustrated embodiments for the distal end 203 of the transparent tube 207 include a smooth closed end 254 (e.g., shown in FIG. 12A) and a flat closed end 255 (e.g., shown in FIG. 12B). It will be appreciated that other shapes and sizes for the distal end 203 may be applicable for certain types of treatments.

A distance between the interior wall 252 and exterior wall 253 is the thickness of the transparent tube 207 wall. The thickness of the tube 207 is generally uniform and minimized to achieve optimal optical transmittance while maintaining the necessary structural characteristics. In some embodiments, the transparent tube 207 has a 1 mm wall thickness. In some other embodiments, the transparent tube 207 may have smaller or larger thickness depending on the material used to create the transparent tube 207 and/or the application in which the transparent tube 207 is to be used.

In some embodiments, the latch feature 258 is sized and oriented to engage with a latch pin (e.g., similar to or the same as the tube latch pin 17 shown installed onto the beam transmission tube 18 in FIG. 2). The latch feature 258 is configured to secure the transparent tube 207 onto the handheld therapy device 201 and prevent rotation.

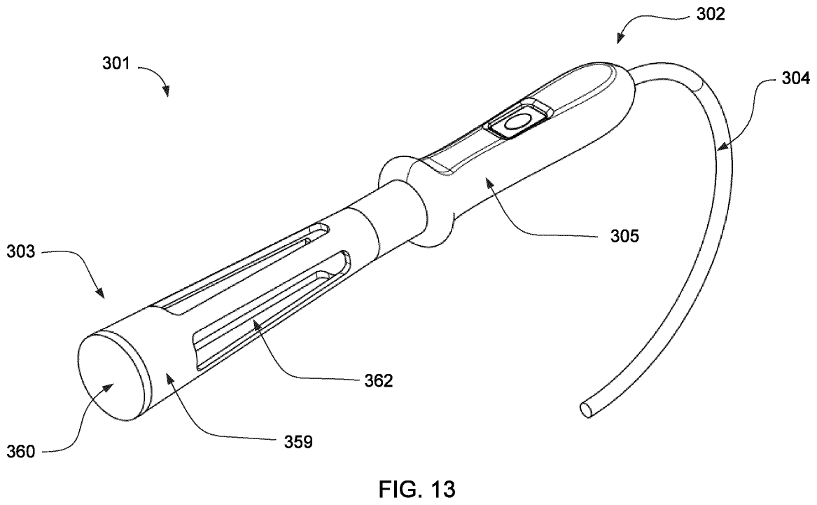

Referring to FIG. 13, a perspective view of a non-cooled surface projection device, shown as a handheld therapy device 301, for the administration of photo biomodulation therapy is shown. The handheld therapy device 301 similarly includes a handle 305 with incoming fiber optic cable 304 at a proximal end 302. The handheld therapy device 301 further includes a beam delivery cone 359 and an emission cap 360 at a distal end 303. In some embodiments, the emission cap 360 is a transparent cover with a convex curvature on the exterior surface and/or is removably attached to the distal end of the beam delivery cone 359. In some embodiments, the non-cool surface projection device may further include a plurality of vent openings 362 in the beam delivery cone 359. The vent openings 362 may allow ambient air to circulate and cool the device.

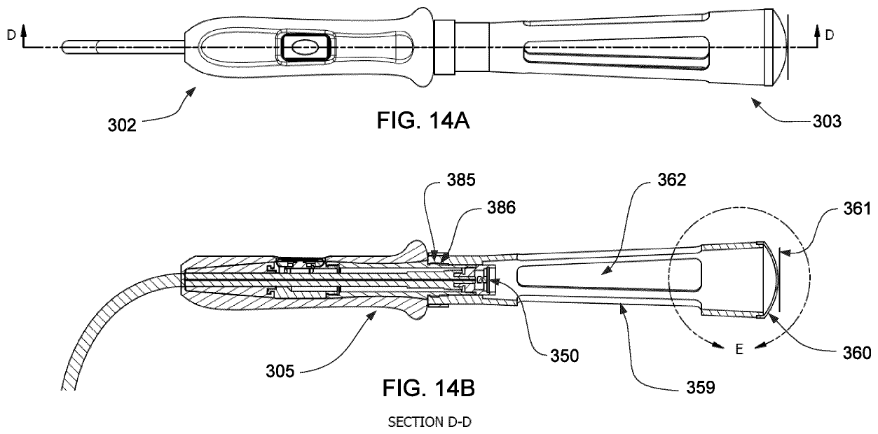

Referring to FIGS. 14A and 14B, a top view (FIG. 14A) of the handheld therapy device 301 is shown, along with a section view (FIG. 14B) of the handheld therapy device 301. As illustrated, the handle 305 at the proximal end 302 includes a mounting interface 386. The mounting interface 86 may be rigidly or removably coupled to the attachment interface 385 of the beam delivery cone 359. When equipped, the vent openings 362 allow air to circulate around the beam forming optics, depicted as double convex lenses 350, and the interior surface of the emission cap 360, which helps cool the device and improve functional efficiency. The double convex lenses 350 produce a beam profile (e.g., similar to or the same as the diverging diffused expanded beam profile 249′ shown in FIG. 10) which is circular and uniformly diffused at a treatment surface 361 adjacent to the emission cap 360 at the distal end 303.

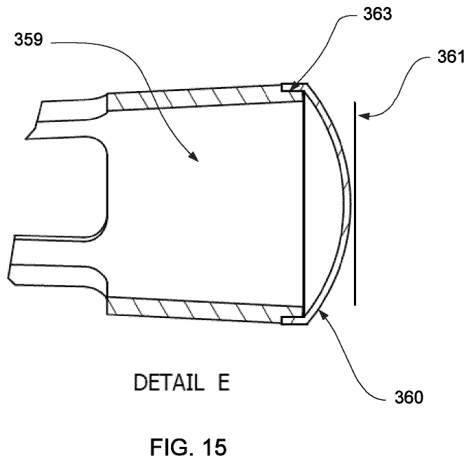

Referring to FIG. 15, a detail view of the handheld therapy device 301 shows the distal end 303 of the handheld therapy device 301 with the beam delivery cone 359 coupled to the emission cap 360 at an emission cap interface 363 and a representative treatment surface 361 adjacent to the emission cap 360. In some embodiments, the emission cap interface 363 provides a removable coupling between the distal end of the beam delivery cone 359 and the emission cap 360 enabling the emission cap 360 to be replaced between patients. In some embodiments, the emission cap interface 363 may include additional connections beyond the physical structural attachment (e.g., electrical connections) to facilitate the integration of sensors and/or additional design features to facilitate toolless connection and/or removal of the emission cap 360 to the beam delivery cone 359. In some embodiments, the emission cap 360 may be manufactured using transparent disposable plastic or a cleanable glass.

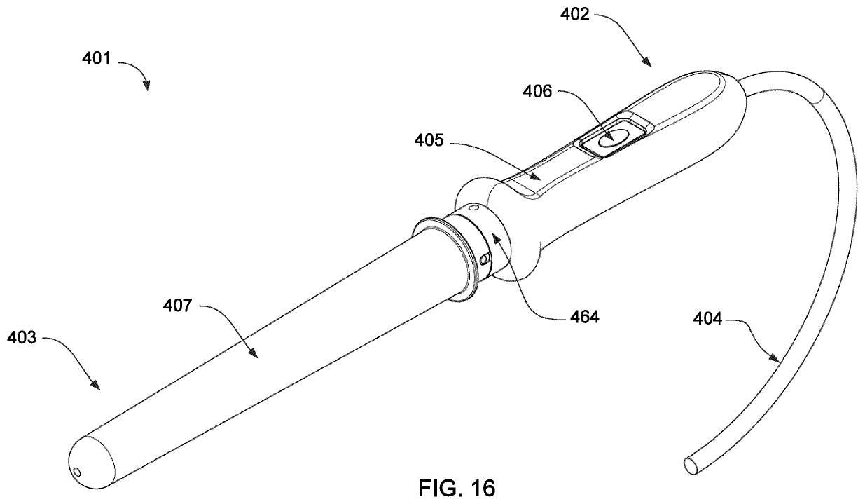

Referring to FIG. 16, a perspective illustration of another encapsulating tube device, shown as a handheld therapy device 401, having internal cooling is shown. The handheld therapy device 401 includes a cooling collar 464 at the junction between the handle 405 and the transparent tube 407 near the proximal end 402 of the handheld therapy device 401. In some embodiments, the cooling collar 464 may serve as a connector between an external cooling system (e.g., the external system 111) and an internal area within the transparent tube 407 allowing coolant to circulate within the handheld therapy device 401.

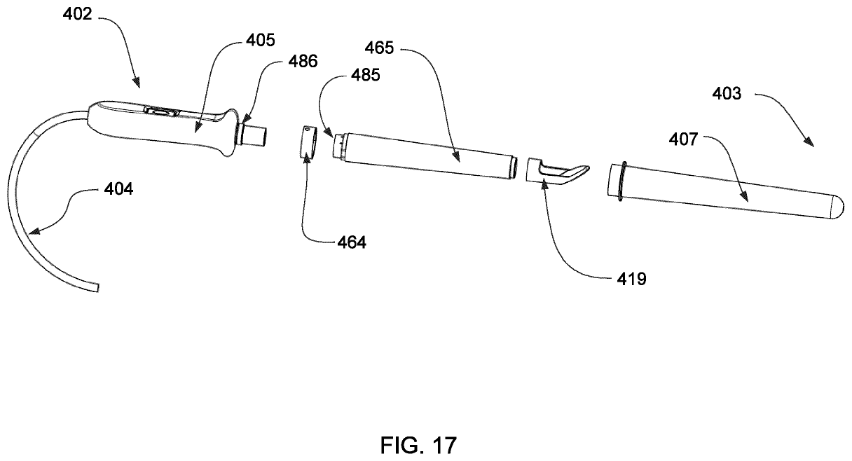

Referring to FIG. 17, a perspective exploded view of the handheld therapy device 401 is shown depicting the various components and interfaces of the handheld therapy device 401. The addition of the cooling collar 464 over the junction between a mounting interface 486 of the handle 405 and an attachment interface 485 at a proximal end of a cooled beam tube 465 facilitates circulation of air or a cooled medium like CO₂ gas within the cooled beam tube 465 to cool the beam reflective end 419, the adjacent portion of the transparent tube 407, and patient contact surfaces.

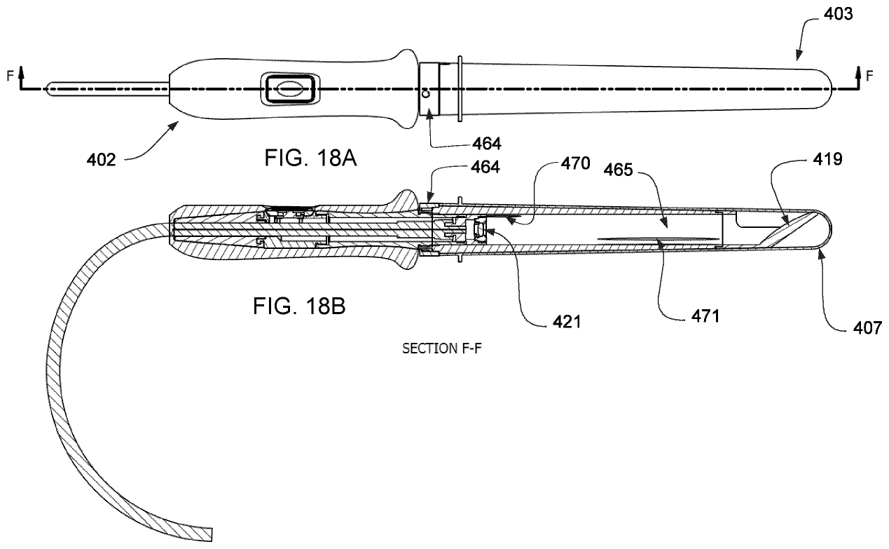

Referring to FIGS. 18A and 18B, a top view (FIG. 18A) and a section view (FIG. 18B) of the handheld therapy device 401 are shown. As illustrated, the cooling collar 464, the beam diffusing lens 421, the cooled beam tube 465, and the beam reflective end 419 are all arranged within the transparent tube 407. The method of operation for the delivery of therapeutic photon energy of the handheld therapy device 401 may be identical or substantially identical to the handheld therapy device 1 depicted in FIGS. 1-6, with the added function of cooling. In some embodiments, coolant (e.g., air, CO₂) flowing into the handheld therapy device 401 at the cooling collar 464 is first incident upon the beam reflective end 419 via an inlet cooled beam tube port 471 at the distal end 403 and then returns via a return cooled beam tube port 470 near the proximal end of the cooled beam tube 465, which additionally allows for circulation of the coolant around the beam diffusing lens 421.

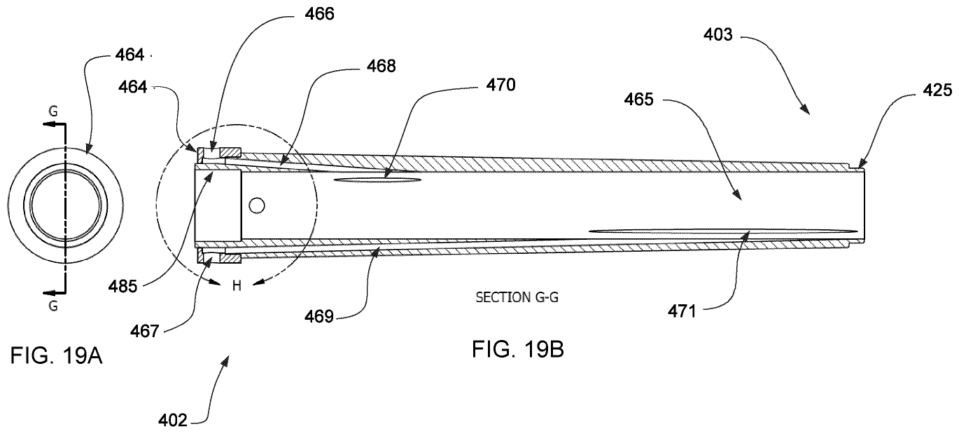

Referring to FIGS. 19A and 19B, an end view (FIG. 19A) and a section view (FIG. 19B) of the cooled beam tube 465 with the cooling collar 464 are shown depicting the various features and characteristics which enable circulating flow of a coolant, typically CO₂ gas, through the handheld therapy device 401. In some embodiments, the cooling collar 464 at the proximal end of the cooled beam tube 465 includes an inlet cooling collar port 467 and a return cooling collar port 466 for corresponding connection to a cooling system (e.g., the external system 111) configured to provide coolant circulation to the handheld therapy device 401. In some embodiments, the cooling collar 464 is directly coupled to the cooled beam tube 465 such that the inlet cooling collar port 467 and the return cooling collar port 466 are connected to the inlet integrated cooling channel 469 and the return integrated cooling channel 468, respectively, within the walls of the cooled beam tube 465. In some embodiments, the junction between the cooling collar 464 and cooled beam tube 465 is configured such that the seams are sealed to prevent leakage of coolant from the device.

The inlet integrated cooling channel 469 travels at a shallow angle through the wall of the cooled beam tube 465 to an inlet cooled beam tube port 471 near the distal end 403 of the cooled beam tube 465. Similarly, the return integrated cooling channel 468 travels at a steeper angle through the wall of the cooled beam tube 465 to a return cooled beam tube port 470 nearer to the proximal end 402 of the cooled beam tube 465. In some embodiments, the proximal end 402 of the cooled beam tube 465 includes attachment interface 485 internal to the cooled beam tube 465 and the cooling collar 464 is externally attached to the cooled beam tube 465. Further, the distal end 403 of the cooled beam tube 465 may include the beam tube to reflector interface 425.

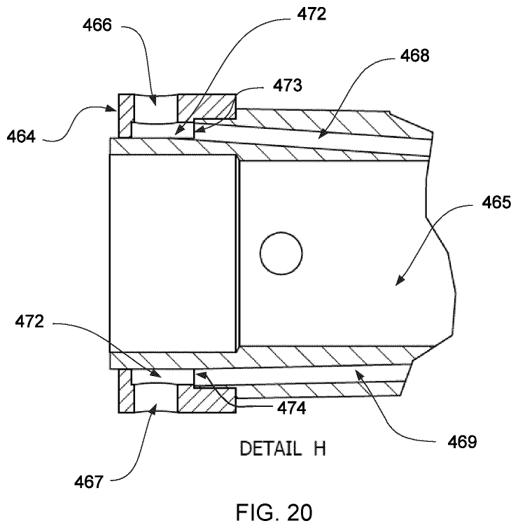

Referring to FIG. 20, a detail view taken along detail line -H- of FIG. 19 is shown depicting the cooling collar 464 on the proximal end of the cooled beam tube 465. The cooling collar 464 is shown with a collar transitional flow cavity 472 connecting the inlet cooling collar port 467 to the inlet integrated cooling channel 469 and a separate collar transitional flow cavity 472 connecting the return integrated cooling channel 468 to the return cooling collar port 466. Coolant flowing to the handheld therapy device 401 from a cooling system (e.g., the external system 111) enters at the inlet cooling collar port 467 and flows through the adjoining collar transitional flow cavity 472 to the inlet integrated cooling channel 469 which delivers coolant to the beam reflective end 419, as depicted in FIG. 18B. The coolant flows circularly through the internal bore of the cooled beam tube 465 to the return integrated cooling channel 468 and through the connected collar transitional flow cavity 472 to the return cooling collar port 466, where a fluidically connected conduit may return the coolant to a cooling system (e.g., the external system 111) to remove the collected heat from the handheld therapy device 401 and circulate cooled coolant back to the inlet cooling collar port 467 of the handheld therapy device 401.

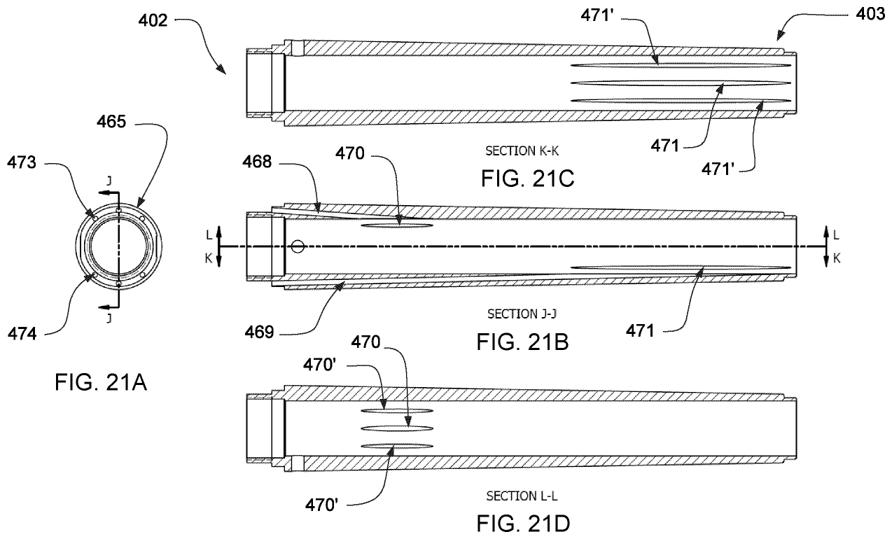

Referring to FIGS. 21A-21D, an end view (FIG. 21A) and various section views (FIGS. 21B-21D) of the cooled beam tube 465 are shown illustrating an inlet integrated channel end hole 474 and a return integrated channel end hole 473. The inlet integrated channel end hole 474 and return integrated channel end hole 473 generally form a path between the inlet integrated cooling channel 469 and the return integrated cooling channel 468 of the cooled beam tube 465 and the inlet cooling collar port 467 and the return cooling collar port 466 the cooling collar 464 via the collar transitional flow cavities 472 shown in FIG. 20.

The inlet integrated cooling channel 469 extends from the inlet integrated channel end hole 474 at the proximal end 402 of the cooled beam tube 465 to the inlet cooled beam tube port 471 at the distal end 403 carrying coolant from an external cooling system (e.g., the external system 111) to a region having the beam reflective end 419 and optical window (e.g., similar to the optical window 105 shown in FIG. 5) inside the transparent tube 407.

The return integrated cooling channel 468 extends from the return integrated cooling channel end hole 473 at the proximal end 402 of the cooled beam tube 465 to the return cooled beam tube port 470 nearer to the proximal end 402 of the cooled beam tube 465 carrying coolant with captured heat from the distal end 403 back to an external cooling system (e.g., the external system 111).

As shown in FIGS. 21C and 21D, in some embodiments, the cooled beam tube 465 comprises a plurality of inlet integrated cooling channels 469 and a plurality of return integrated cooling channels 468 extending to a corresponding inlet cooled beam tube port 471 and a return cooled beam tube port 470 and/or one or more auxiliary inlet cooled beam tube ports 471′ (as shown in FIG. 21C) and one or more auxiliary return cooled beam tube ports 470′ (as shown in FIG. 21D).

In some embodiments, the cooled beam tube 465 may be made of a thermally insulating material to minimize the loss of heat through the inlet integrated cooling channel 469 and the return integrated cooling channel 468. In other embodiments, the cooled beam tube 465 may be made of a metallic material (e.g., aluminum or stainless steel) and/or may require the addition of insulating materials or coatings to improve system efficiency and minimize losses.

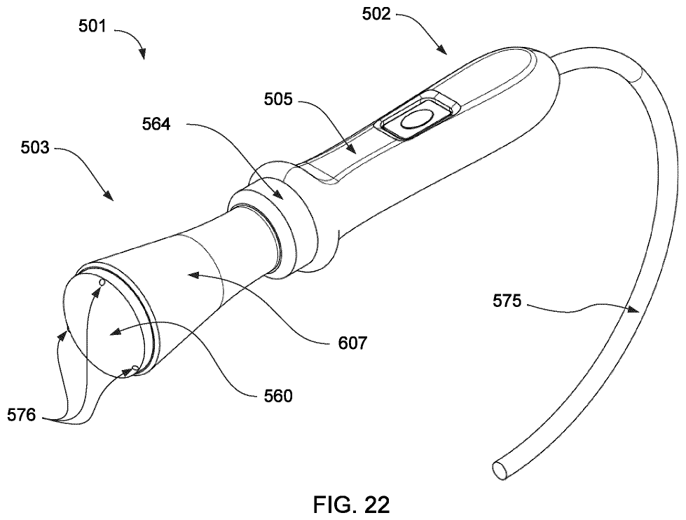

Referring to FIG. 22, a perspective view of a cooled surface projection device, shown as a handheld therapy device 501, having an integrated cooling system is shown. The handheld therapy device 501 has an umbilical cable 575 entering a handle 505 at a proximal end 502 and interfacing with a cooling collar 564 and a cooled beam delivery cone 607. The handheld therapy device 501 additionally includes one or more temperature sensors 576 at a distal end 503 of the handheld therapy device 501. In some embodiments, the temperature sensors 576 project through the exterior surface of the emission cap 560, such that they are able to measure a temperature of patient tissue in contact with the emission cap 560.

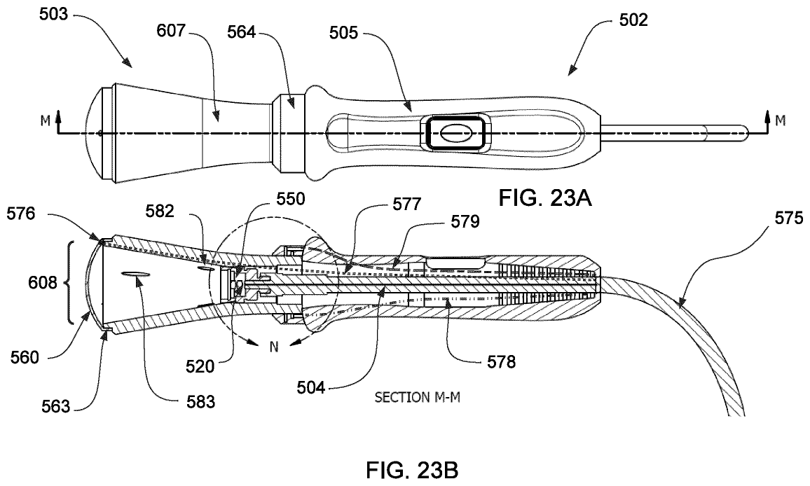

Referring to FIGS. 23A and 23B, a top view (FIG. 23A) and a section view (FIG. 23B) of the handheld therapy device 501 are shown depicting the various integrated features contained within the handheld therapy device 501. An umbilical cable 575 of the handheld therapy device 501 may be a cable assembly comprising a fiber optic cable 504, an inlet cooling conduit 578, a return cooling conduit 579, and temperature sensor wires 577 providing operational connections between the handheld therapy device 501 and an integrated control system with the therapeutic photon energy source, the cooling system or apparatus, and/or various other electronics and controls (e.g., each of which may be incorporated within or included as part of the external systems 111).

The inlet cooling conduit 578 extends from the umbilical cable 575 at the proximal end 502 of the handheld therapy device 501, through the handle 505 where it is in fluid communication with the cooling collar 564, and facilitates the direct flow and delivery of coolant to the distal end 503 of the cooled beam delivery cone 607, where coolant flows from an inlet cooled beam cone port 583 and flows incident upon an internal surface of the emission cap 560 facilitating cooling of the emission cap 560 and adjacent patient tissue surfaces, thereby maximizing or otherwise improving therapeutic efficacy.

A return cooled beam cone port 582 is located nearer to a proximal end of the cooled beam delivery cone 607 and serves as a collector for coolant that carries heat away from the emission cap 560 at the distal end 503 and back to the integrated cooling system (e.g., the external system 111) via fluidic pathways through the cooling collar 564, the return cooling conduit 579, and the umbilical cable 575.

The handheld therapy device 501 has an optical window 608 defined by a diameter of an opening of the distal end 503 of the cooled beam delivery cone 607 and corresponding emission cap 560 diameter in conjunction with beam forming optics, depicted as double convex lenses 550, to produce a diffuse beam profile of a predetermined size at a specified distance from the emission point 520.

The temperature sensor(s) 576, which may be implemented, for example, as one or more thermocouples, are connected to an integrated control system via temperature sensor wires 577 extending from the temperature sensor(s) 576, which may be in or on the cooled beam delivery cone 607 and/or within the handle 505, as part of the umbilical cable 575 to a suitable connection at the control system (e.g., the external system 111). In some embodiments, a sensing surface of one or more of the temperature sensor(s) 576 is positioned on a periphery of the distal end 503 of the cooled beam delivery cone 607 such that it is exposed to an ambient environment tangent to the surface of the emission cap 560.

A cap interface 563 between the cooled beam delivery cone 607 and emission cap 560 may include clearances for temperature sensor(s) 576 to extend through the emission cap 560 from within the cooled beam delivery cone 607. In some embodiments, the cap interface 563 includes connection features between the temperature sensor wires 577 and the temperature sensor 576 effectively allowing for selective decoupling of the sensor(s) 576 from the temperature sensor wires 577, allowing the emission cap 560 to have the temperature sensor(s) 576 integrated therein, and allowing connections of the temperature sensor(s) 576 to the temperature sensor wires 577 to be made at the cap interface 563 when the emission cap 560 is installed onto the cooled beam delivery cone 607.

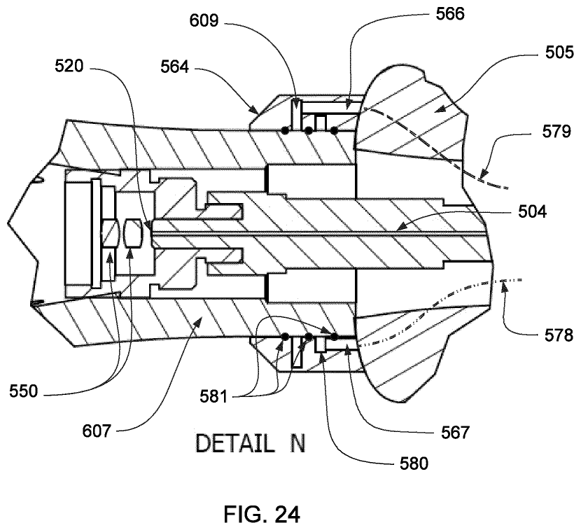

Referring to FIG. 24, a detail view of the handheld therapy device 501 depicts various components and interfaces at the region between the handle 505 and cooled beam delivery cone 607. The cooling collar 564 is shown with inlet cooling collar port 567, which intersects an inlet collar channel 580, and the return cooling collar port 566, which intersects return collar channel 609, which collectively allow coolant to flow into and out of the handheld therapy device 501. Coolant flows into the device from and external cooling system (e.g., the external system 111), via inlet cooling conduit 578, entering the cooling collar 564 at the inlet cooling collar port 567 and returns to the external cooling system from the handheld therapy device 501 via the return cooling conduit 579 connected to the return cooling collar port 566 of the cooling collar 564.

The inlet cooling collar port 567 and return cooling collar port 566 and corresponding intersecting inlet collar channel 580 and return collar channel 609 are sealed between the cooling collar 564 and cooled beam delivery cone 607 by collar channel seal(s) 581, which may, in some embodiments, be o-rings. The inlet collar channel 580 and return collar channel 609 facilitate the flow between the cooling collar 564 and cooled beam delivery cone 607.

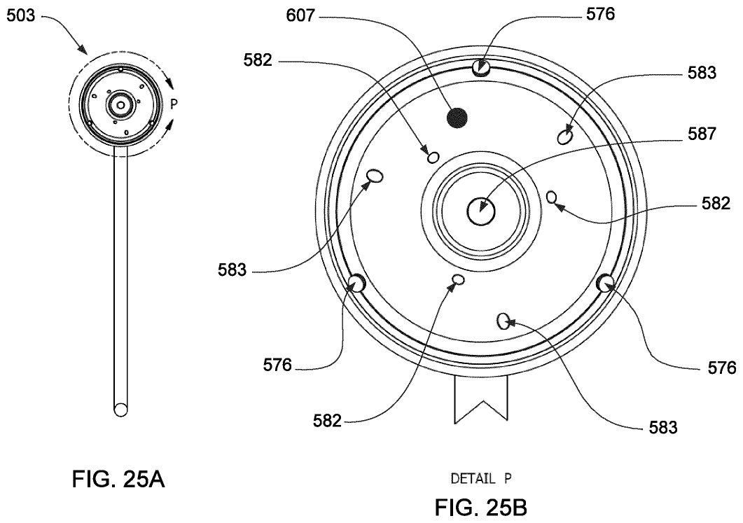

Referring to FIGS. 25A and 25B, a distal end view (FIG. 25A) and a detail view (FIG. 25B) of the handheld therapy device 501 are shown depicting the radial arrangement of the inlet cooled beam cone port 583 and the return cooled beam cone port 582 of the cooled beam delivery cone 607, as well as the temperature sensors 576. In the illustrated embodiment shown in FIG. 25, a plurality of the inlet cooled beam cone ports 583, return cooled beam cone ports 582, and temperature sensors 576 are arranged and evenly distributed circumferentially around and radially concentric with an axis of the beam diffusing optics 587 (e.g., a first circle formed by the plurality of the inlet cooled beam cone ports 583, a second circle formed by the plurality of return cooled beam cone ports 582, and a third circle formed by the temperature sensors 576 are all radially concentric with the axis of the beam diffusing optics 587). In other embodiments, other arrangements of the inlet cooled beam cone ports 583, the return cooled beam cone ports 582, and the temperature sensors 576 may be implemented within the handheld therapy device 501.

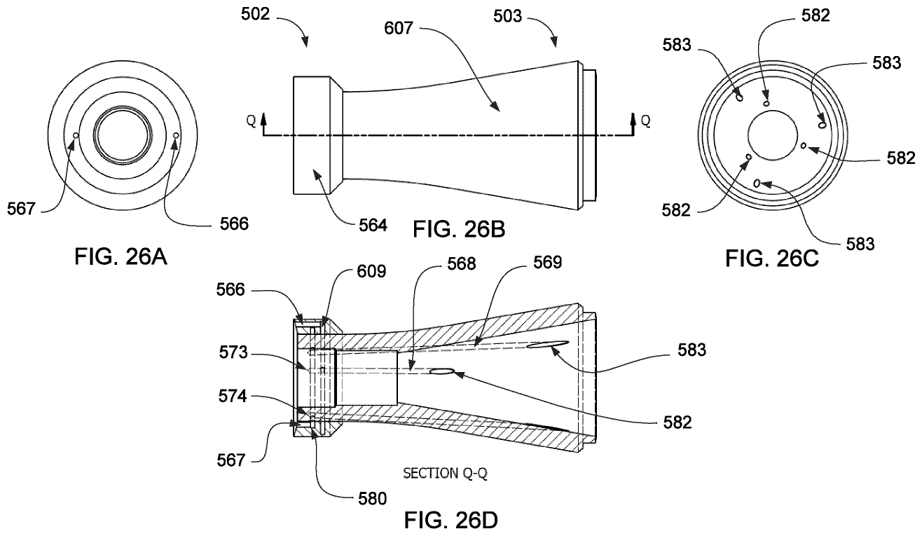

Referring to FIGS. 26A-26D, front (FIG. 26A), side (FIG. 26B), and rear (FIG. 26C) views, as well as a section view (FIG. 26D), of the cooled beam delivery cone 607 and connected cooling collar 564 are shown depicting coolant flow paths between the inlet cooling collar port 567 and the return cooling collar port 566 and the inlet cooled beam cone port 583 and return cooled beam cone port 582. During use, coolant entering the handheld therapy device 501 through the inlet cooling collar port 567 flows into the inlet collar channel 580, which may be a groove around the inside of the cooling collar 546. The inlet collar channel 580 intersects with one or more inlet integrated cooling channel(s) 569, allowing the coolant to flow through the inlet integrated cooling channel 569 to the inlet cooled beam port 583 near the distal end 503 of the cooled beam delivery cone 607. Similarly, one or more return cooled beam port(s) 582 located nearer to the proximal end 502 of the cooled beam delivery cone 607 collect coolant and allow it to flow through the return integrated cooling channel 568 to where it intersects the return collar channel 609 and connects to the return cooling collar port 566 from which the coolant can return to the cooling system (e.g., the external system 111).

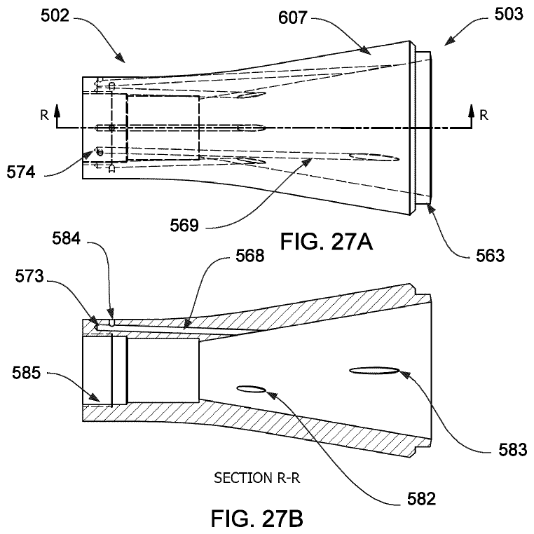

Referring to FIGS. 27A and 27B, a side view (FIG. 27A) and a section view (FIG. 27B) the cooled beam delivery cone 607 are shown depicting integrated flow cavities within walls of the cooled beam delivery cone 607 that facilitate flow of coolant between the cooled beam delivery cone 607 and the cooling collar 564 depicted in FIG. 26B. FIG. 27B illustrates the flow path through the cooled beam delivery cone 607 for the return flow of coolant carrying heat from the handheld therapy device 501 to integrated control systems (e.g., the external system 111). The return cooled beam cone port 582 and connected return integrated cooling channel 568 extends to the proximal end 502 of the cooled beam delivery cone 607 and terminates at the return integrated channel end 573. A cooling collar interface port 584 is a hole intersecting the return integrated cooling channel 568 and aligned to the return collar channel 609 illustrated in FIG. 27B.

The inlet flow path follows a similar implementation scheme with the incoming flow of coolant configured to enter the cooled beam delivery cone 607 at the cooling collar interface port 584 aligned with the inlet integrated cooling channel 569 and exits the inlet integrated cooling channel 569 at the inlet cooled beam cone port 583 at the distal end 503 of the cooled beam delivery cone 607. The cooled beam delivery cone 607 further includes an attachment interface 585 at the proximal end 502 configured to enable attachment to the handle 505 and a cap interface 563 at the distal end 503 configured for attachment to the emission cap 560.

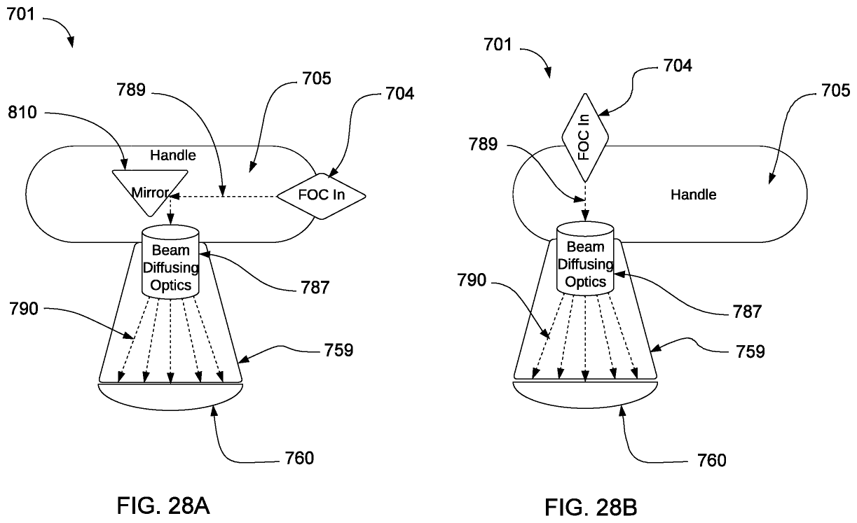

Referring to FIGS. 28A and 28B, two configurations of a surface projection device, shown as handheld therapy device 701, are shown. While the handheld therapy devices shown in FIGS. 12, 13, 22 and 23 have been depicted as long slender devices, these configurations of the handheld therapy device 701 each include a handle 705 whose longitudinal axis is perpendicular to a beam delivery cone 759 axis of its beam diffusing optics 787, produced diffuse beam 790, and emission cap 760.

In the first configuration (FIG. 28A), a fiber optic cable (FOC) 704 is connected to the handle 705 at one end and coherent light 789 emitted from the end of the FOC 704 is reflected off an angled mirror 810 and then through the beam diffusing optics 787. In the second configuration (FIG. 28B), the FOC 704 is connected to handle 705 such that the coherent light 789 is emitted in axial alignment with the beam diffusing optics 787 near one end of the handle 705.

These configurations for the handle 705 of the handheld therapy device 701 and the addition of the mirror 810 enable different ergonomic implementations that may be easier to manipulate and/or operate during certain procedures.

In the illustrated embodiment shown in FIG. 28A, the angled mirror 810 is depicted as having approximately a 45 degree angle with respect to the coherent light 789 emitted from the end of the FOC 704. However, in other embodiments, the angled mirror 810 could be at any angle producing a corresponding angular deviation between the longitudinal axis of the handle 705 and the emission axis of the beam diffusing optics 787 and diffuse beam 790.

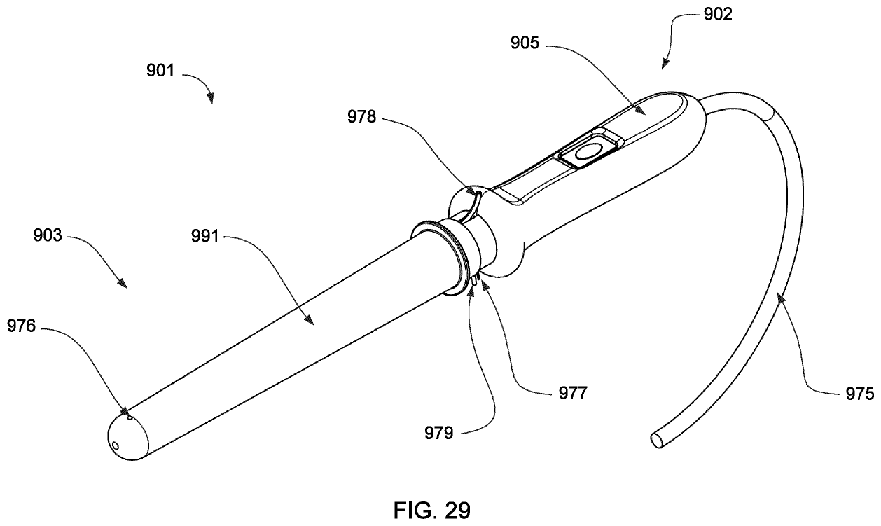

Referring to FIG. 29, a perspective illustration of another embodiment of a cooled tube device, shown as a handheld therapy device 901, having a temperature sensor 976 and a transparent tube 991 configured to integrate with the temperature sensor 976. In some instances, the handheld therapy device 901 further includes an inlet cooling conduit 978, a return cooling conduit 979, and temperature sensor wires 977 exiting the device adjacent to the handle 905 for connection to external cooling and measurement systems (e.g., the external systems 111). In some other instances, the inlet cooling conduit 978, the return cooling conduit 979, and the temperature sensor wires 977 could pass through the handle 905 and extend through an umbilical cable 975 to the integrated control system (e.g., the external system 111), as desired for a given application.

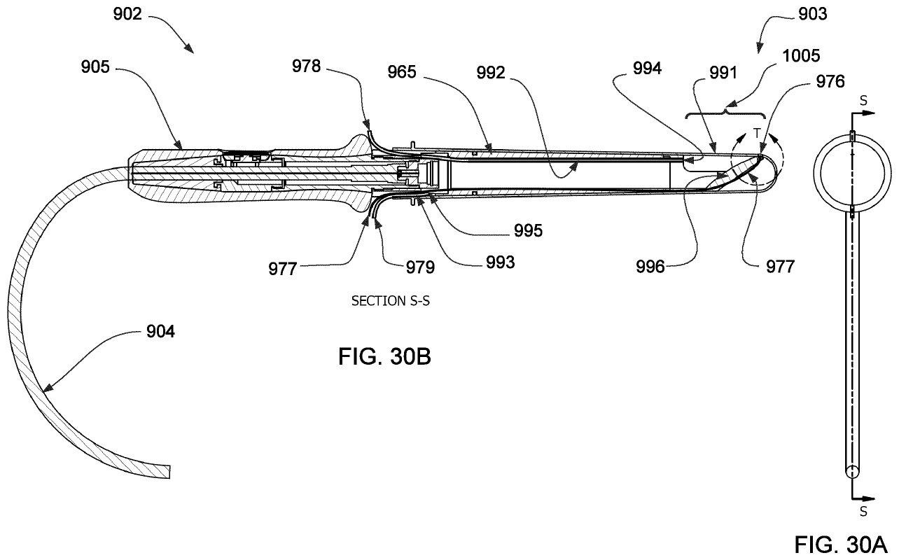

Referring to FIGS. 30A and 30B, an end view (FIG. 30A) and a section view (FIG. 30B) of the handheld therapy device 901 are shown illustrating the location, routing, and alignment of various components and features of handheld therapy device 901.

FIG. 30B shows a coolant flow path entering the handheld therapy device 901 via an inlet cooling conduit 978 at a proximal end of a cooled beam tube 965 and continuing in fluid communication with an internal cooling inlet conduit 992 within the cooled beam tube 965. Coolant exits the internal cooling inlet conduit 992 at the cooling inlet nozzle 994 near the distal end 903 of the cooled beam tube 965 where it is directed across an interior surface of the optical window 1005 region of the transparent tube 991. Flowing coolant travels across the surface of the beam reflective end 996 with the temperature sensor 976 and flows through a void space within the cooled beam tube 965 to a cooling return collector 995 near the proximal end of the cooled beam tube 965. The cooling return collector 995 may be integrated or fluidically connected to the internal cooling return conduit 993 and the return cooling conduit 979.

Temperature sensor 976 is in thermal communication with an external surface of the transparent tube 991. The temperature sensor wires 977 are routed through the cavity of the cooled beam tube 965 between the temperature sensor 976 at the distal end 903 adjacent to the optical window 1005 and the beam reflective end 996 and along the surface or within the walls of the cooled beam tube 965 to the exit at the proximal end 902 of the cooled beam tube 965 adjacent to the handle 905.

Internal cooling inlet conduit 992 and internal cooling return conduit 993 may be formed in a single- or multi-wall insulating assembly to prevent thermal losses between the coolant flowing through the internal cooling inlet conduit 992 and internal cooling return conduit 993 and the cooled beam tube 965, the ambient environment, or otherwise with the handheld therapy device 901 to optimize heat transfer and cooling effects.

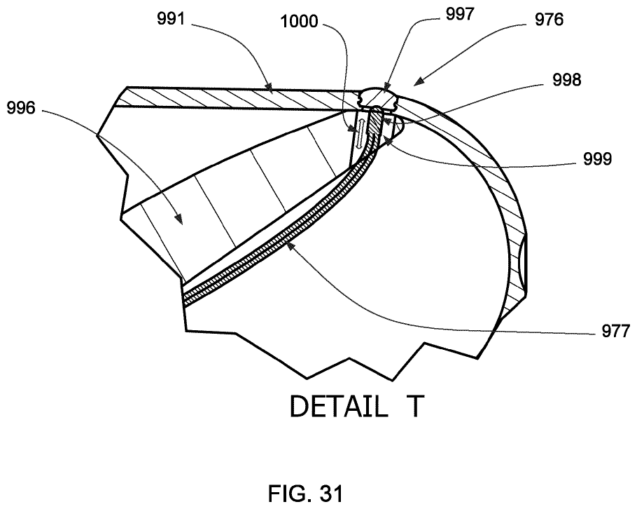

Referring to FIG. 31, a detail view of the handheld therapy device 901 is shown depicting an example configuration of the temperature sensor 976 integration within the transparent tube 991 and beam reflective end 996. In some embodiments, a thermal contact 998 is in physical thermally conductive contact with a thermal sensor cap 997 integrated within the structure of the transparent tube 991. In some embodiments, the thermal sensor cap 997 is constructed or made of a material with high thermal conductivity, such as copper, gold, silver, or any other suitable thermally conductive material. The thermal contact 998 may be mounted within a thermal insulator 999 installed into a cavity at the distal tip of the beam reflective end 996 to prevent heat from the beam reflective end 996 offsetting the reading from the external surface in contact with the handheld therapy device 901. In some embodiments, the temperature sensor 976 integration includes, in some embodiments, a compliant element 1000 within the assembly of the thermal contact 998 and thermal insulator 999. The compliant element 1000, which may be, e.g., a spring, provides a positive contact force between the thermal contact 998 and thermal sensor cap 997 without requiring physical coupling and facilitating the removal and replacement of the transparent tube 991. The temperature sensor wires 977 extend from the thermal insulator 999 where they are connected to the thermal contact 998. In some embodiments, the thermal insulator 999 is itself constructed or made of a material with low thermal conductivity (e.g., plastic or ceramic). Accordingly, heat from (e.g., indicative of a temperature of) a patient's tissue on an external surface of the thermal sensor cap 997 may be conducted through the thermal sensor cap 997 (e.g., via the thermally conductivity of the thermal sensor cap 997) and sensed by the temperature sensor 976 while heat (or cooling from circulating coolant) within the handheld therapy device 901 does not affect a sensor reading of the temperature sensor 976.

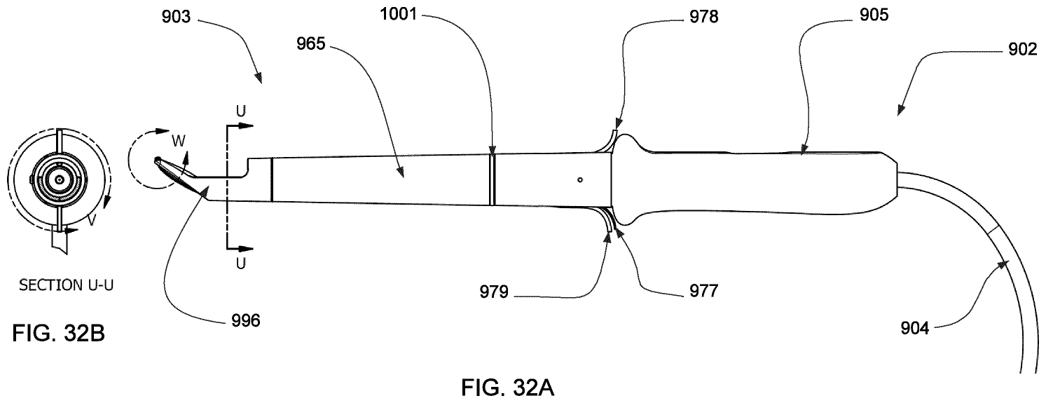

Referring to FIGS. 32A and 32B, a side view (FIG. 32A) and a section view (FIG. 32B) of the handheld therapy device 901 are shown without the transparent tube 991. As illustrated, a seal, shown as an o-ring seal 1001, may be arranged along an outside surface of the cooled beam tube 965 approximately midway along a length of the handheld therapy device 901 between the proximal end 902 and the distal end 903. The o-ring seal 1001 may thus provide a seal between the cooled beam tube 965 and the transparent tube 991 (e.g., shown in FIG. 30).

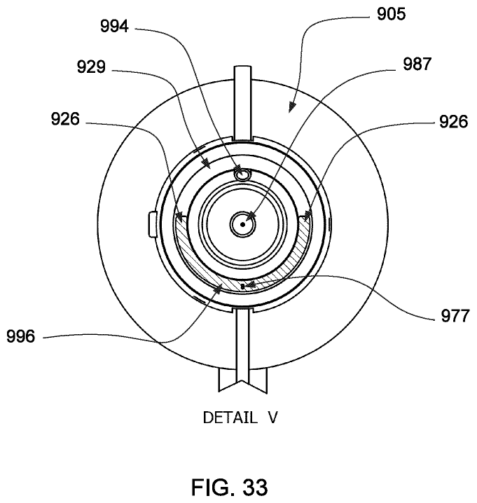

Referring to FIG. 33, a detail section view of the handheld therapy device 901 is shown depicting a plane midway across the beam reflective end 996 perpendicular to a longitudinal axis of the handheld therapy device 901 aligned with the beam diffusing optics 987 and looking from the distal end 903 towards the proximal end 902 of the handle 905.

The beam reflective end 996 is shown with optical window wings 926 in section and an optical window proximal edge 929. At a top position is the cooling inlet nozzle 994 depicted as elliptical in shape to reduce the cross section of interference of the cooling inlet nozzle 994 to the diffuse light beam coming out of the beam diffusing optics 987 before being reflected by the beam reflective end 996. As best shown in FIGS. 30B and 33, the temperature sensor wires 977 are shown within the body of the beam reflective end 996 and the cooled beam tube 965.

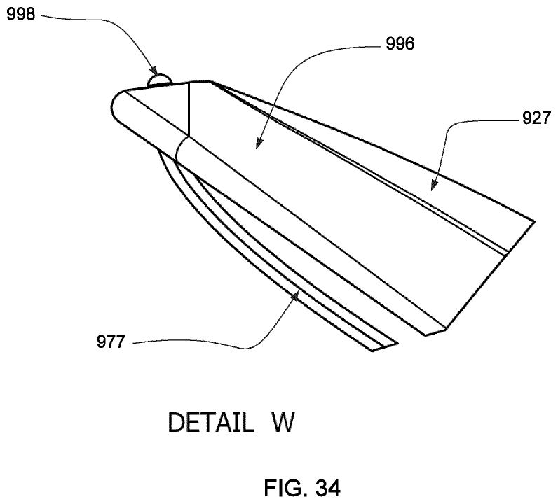

Referring to FIG. 34, a detail view of a distal tip of the beam reflective end 996 of the handheld therapy device 901 is shown. As shown, in some embodiments, the thermal contact 998 is positioned distally beyond the end of the convex reflective surface 927 with the temperature sensor wires 977 extending behind the beam reflective end 996. The positioning of the thermal contact 998 is such that it does not interfere with the optical transmission of the therapeutic photon energy but is sufficiently close to the convex reflective surface 927 to measure reliable temperature data for adjacent patient tissues being treated.

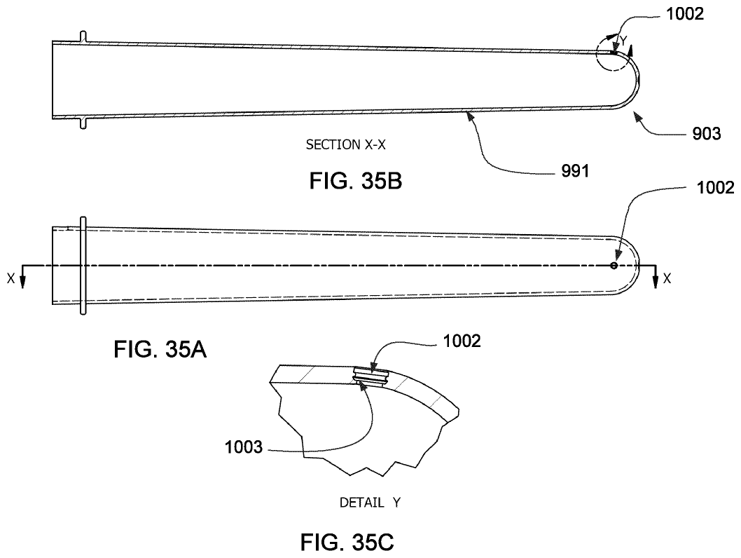

Referring to FIGS. 35A-35C, a side view (FIG. 35A), a cross-section view (FIG. 35B), and a detail view (FIG. 35C) of the transparent tube 991 are shown illustrating example details of the temperature sensor integration. At the distal end 903 of the transparent tube 991 is a tube temperature sensing port 1002. The tube temperature sensing port 1002 is a passage through the wall of the transparent tube 991 with tube temperature port features 1003 which may be configured to match the physical geometries of the thermal sensor cap 997 shown in FIG. 31.

In some embodiments, the tube temperature sensing port 1002 may be molded into the transparent tube 991. In some embodiments, the tube temperature sensing port 1002 may be added as an independent operation for installation of a thermal sensor cap 997 after the transparent tube 991 is manufactured. In some embodiments, the thermal sensor cap 997 may be molded with the transparent tube 991, thereby producing a single part assembly of the transparent tube 991 and thermal sensor cap 997 located in the resulting tube temperature sensing port 1002 with tube temperature port features 1003.

For the purposes of this disclosure, it is to be assumed that the components and features described herein are not bound by any particular material or manufacturing processes. The depicted embodiments may lend themselves to a particular manufacturing process, however it is to be known that the functions can be achieved using various processes and corresponding design changes to accommodate process limitations or advantages. The construction of components is agnostic to material choice. Materials may be selected based on physical structure, electrical, or thermal characteristics and can be different for each embodiment as dictated by the application and design preferences.

Further, while certain embodiments shown and described herein include specific features, it will be appreciated that features of certain embodiments described herein may be implemented or otherwise utilized in other embodiments described herein. Similarly, features shown in certain embodiments may be omitted, as desired for a given application or use-case scenario. Each of these variations is contemplated in the present disclose.

Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, Z, X and Y, X and Z, Y and Z, or X, Y, and Z. Thus, such conjunctive language is not generally intended to imply that certain embodiments require at least one of X, at least one of Y, and at least one of Z to each be present.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed:

1. A handheld therapy device comprising:
a handle having a fiber optic cable and a diffusing light assembly, the fiber optic cable extending through the handle and configured to transmit a beam of coherent light through the diffusing light assembly;
a beam transmission tube attached to the handle, the beam transmission tube configured to receive diffused light from the diffusing light assembly and allow the diffused light to pass through the beam transmission tube in an axial direction;
a beam reflective end removably attached to a distal end of the beam transmission tube, the beam reflective end configured to receive the diffused light passed through the beam transmission tube and to reflect the diffused light in a non-axial direction; and
a transparent tube removably attached to the handle and at least partially enveloping the beam transmission tube, the transparent tube being at least partially transparent to allow the diffused light reflected in the non-axial direction off of the beam reflective end to pass therethrough.

2. The handheld therapy device of claim 1, wherein the transparent tube includes an exterior rib configured to at least one of secure the transparent tube on the handle, serve as a depth reference for treatment, or provide a physical depth limit for treatment.

3. The handheld therapy device of claim 1, wherein the beam transmission tube includes one or more coolant flow channels configured to direct coolant onto the beam reflective end.

4. The handheld therapy device of claim 3, wherein the one or more coolant flow channels are integrated into a circumferential wall of the beam transmission tube.

5. The handheld therapy device of claim 3, further comprising a cooling collar configured receive the coolant from a coolant source and to direct the coolant into the one or more coolant flow channels of the beam transmission tube.

6. The handheld therapy device of claim 1, wherein the beam transmission tube and the beam reflective end collectively form an optical window between a proximal optical window edge of the beam transmission tube, a pair of optical window wings of the beam transmission tube, and the beam reflective end.

7. The handheld therapy device of claim 1, wherein the transparent tube includes a temperature sensing port having a thermal sensor cap in contact with a temperature sensor located within the transparent tube, the thermal sensor cap configured to conduct heat from a patient tissue through the transparent tube to the temperature sensor.

8. The handheld therapy device of claim 1, wherein the beam reflective end has a profile defined by a first radius of curvature in a first direction and a second radius of curvature in a second direction, the first radius of curvature being different than the second radius of curvature, and the first direction being perpendicular to the second direction.

9. The handheld therapy device of claim 8, wherein, when the diffused light has a Gaussian beam profile having a Gaussian distribution of energy prior to being reflected by the beam reflective end, the beam reflective end is configured to reflect the diffused light to produce a uniform beam profile having a uniform distribution of energy.

10. The handheld therapy device of claim 1, wherein the beam transmission tube is removably attached to the handle.

11. A handheld therapy device comprising:
a handle having a fiber optic cable and a diffusing light assembly, the fiber optic cable extending through the handle and configured to transmit a beam of coherent light through the diffusing light assembly;
a beam delivery cone removably attached to the handle, the beam delivery cone configured to receive diffused light from the diffusing light assembly and allow the diffused light to pass axially through the beam delivery cone; and
a transparent emission cap removably attached to a distal end of the beam delivery cone, the transparent emission cap being at least partially transparent to allow the diffused light passing axially through the beam delivery cone to pass therethrough.

12. The handheld therapy device of claim 11, wherein the beam delivery cone includes a plurality of vent openings configured to allow ambient air to circulate within the beam delivery cone.

13. The handheld therapy device of claim 11, wherein the beam delivery cone includes one or more coolant flow channels configured to direct coolant onto the transparent emission cap.

14. The handheld therapy device of claim 13, wherein the one or more coolant flow channels are integrated into a circumferential wall of the beam delivery cone.

15. The handheld therapy device of claim 13, further comprising a cooling collar configured receive the coolant from a coolant source and to direct the coolant into the one or more coolant flow channels of the beam delivery cone.

16. The handheld therapy device of claim 11, wherein the transparent emission cap includes a temperature sensor configured to detect a temperature of patient tissue adjacent to the transparent emission cap.

17. A handheld therapy device comprising:
a handle having a fiber optic cable and a diffusing light assembly, the fiber optic cable extending through the handle and configured to transmit a beam of coherent light through the diffusing light assembly;
a beam passing component removably attached to the handle, the beam passing component being at least partially hollow and configured to receive diffused light from the diffusing light assembly and allow the diffused light to pass through the beam passing component in an axial direction; and
a transparent emission component removably attached to one of the handle or a distal end of the beam passing component, the transparent emission component being at least partially transparent to allow the diffused light passing axially through the beam passing component to pass therethrough.

18. The handheld therapy device of claim 17, wherein the beam passing component is a beam transmission tube and the transparent emission component is a transparent tube removably attached to the handle and at least partially enveloping the beam transmission tube.

19. The handheld therapy device of claim 18, further comprising a beam reflective end removably attached to a distal end of the beam transmission tube, the beam reflective end configured to receive the diffused light passed through the beam transmission tube and to reflect the diffused light in a non-axial direction.

20. The handheld therapy device of claim 17, wherein the beam passing component is a beam delivery cone and the transparent emission component is a transparent emission cap removably attached to a distal end of the beam delivery cone.
